(19) United States
(12) Patent Application Publication
(10) Pub. No.: US 2025/0136603 A1
(43) Pub. Date: May 1, 2025

(54) METHODS AND INTERMEDIATES FOR PREPARING ANTIVIRAL PRODRUGS

(71) Applicant: Gilead Sciences, Inc., Foster City, CA (US)

(72) Inventors: Andrei Chtchemelinine, San Mateo, CA (US); Gregory Cizio, San Mateo, CA (US); Christopher Discolo, Redwood City, CA (US); John Enquist, San Mateo, CA (US); Lucas Man, Burlingame, CA (US); Sean T. Neville, San Mateo, CA (US); Keshab Sarma, Sunnyvale, CA (US); Bing Shi, Redwood City, CA (US); Leon X. Sun, Pleasanton, CA (US); Kevin S. Williamson, San Mateo, CA (US)

(21) Appl. No.: 18/754,347

(22) Filed: Jun. 26, 2024

Related U.S. Application Data
(60) Provisional application No. 63/523,555, filed on Jun. 27, 2023.

(51) Int. Cl. C07D 473/40; B01J 31/00; B01J 31/02; C07D 307/33; C12P 7/04

(52) U.S. Cl. CPC C07D 473/40; B01J 31/006; B01J 31/0244; B01J 31/0284; B01J 31/0287; C07D 307/33; C12P 7/04; B01J 2231/643; B01J 2531/821

(57) ABSTRACT

The present invention is related to processes for preparing antiviral prodrugs, co-crystals, solvents, salts or combinations thereof, and related synthetic intermediate compounds.

### CROSS-REFERENCE TO RELATED APPLICATIONS

This application claims the benefit under 35 U.S.C. § 119 (e) of U.S. Provisional Application No. 63/523,555, filed on Jun. 27, 2023, which is hereby incorporated herein by reference in its entirety for all purposes.

### TECHNICAL FIELD

The present invention relates to methods and intermediates for the synthesis of antiviral prodrugs, co-crystals, solvates, salts, or combinations thereof, and related synthetic intermediate compounds.

### BACKGROUND

The present disclosure relates generally to the field of organic synthetic methodology for the preparation of antiviral prodrugs and their synthetic intermediates.

Positive-single stranded RNA viruses comprising the Retroviridae family include those of the subfamily Orthoretrovirinae and genera Alpharetrovirus, Betaretrovirus. Gammaretrovirus, Deltaretrovirus, Epsilonretrovirus, Spumavirus, and Lentivirus, which cause many human and animal diseases. Among the Lentivirus, HIV-1 infection in humans leads to depletion of T helper cells and immune dysfunction, producing immunodeficiency and vulnerability to opportunistic infections.

One approach to treating HIV-I infection is by administering NRTTIs, NRTTIs inhibit HIV-1 reverse transcriptase and, because reverse transcriptase function is essential for viral replication and production of viral proteins. NRTTIs can be effective against HIV-1 infection. Curr Opin HIV AIDS. 2018 July; 13(4): 294-299. HIV treatments, however, have historically led to the emergence of HIV strains that are resistant to current therapies. Expert Opin Emerg Drugs. 2018 June; 23(2): 149-157. Therefore, there is an ongoing need to discover new antiretroviral agents and to develop methods for their preparation and purification.

U.S. patent application Ser. No. 17/642,552 discloses novel compounds useful for treating HIV infection. One specific compound identified therein is a compound of formula I:

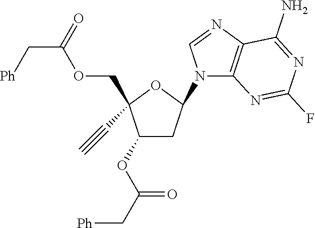 [SMILES: C#C[C@]1(COC(=O)Cc2ccccc2)O[C@@H](n2cnc3c(N)nc(F)nc32)C[C@@H]1OC(=O)Cc1ccccc1]

There is currently a need for improved synthetic methods and intermediates that can be used to prepare the compound of formula I and co-crystals, solvates, salts, and combinations thereof. There is also a need for improved methods for preparing intermediate compounds that can be used to prepare the compound of formula I and its co-crystals, solvates, salts, and combinations thereof. The improved methods and intermediates may reduce the cost, time, and/or the amount of waste associated with the existing methods for preparing the compound of formula I and co-crystals, solvates, salts, and combinations thereof. The methods disclosed herein meet these and other needs.

### SUMMARY

The present disclosure provides, inter alia, processes of preparing a compound of formula I:

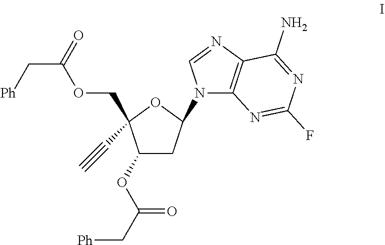 [SMILES: C#C[C@]1(COC(=O)Cc2ccccc2)O[C@@H](n2cnc3c(N)nc(F)nc32)C[C@@H]1OC(=O)Cc1ccccc1]

or a co-crystal, solvate, salt, or combination thereof;
comprising reacting a compound of formula XVII:

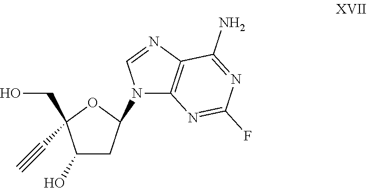 [SMILES: C#C[C@]1(CO)O[C@@H](n2cnc3c(N)nc(F)nc32)C[C@@H]1O]

or a co-crystal, solvate, salt, or combination thereof, with a compound of formula XVIII:

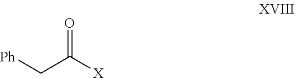

wherein X is selected from halo, —OCOCH₂Ph, and OH, and
a base,
to provide the compound of formula I or a co-crystal, solvate, salt, or combination thereof.

The present disclosure further provides a process for preparing a compound of formula I:

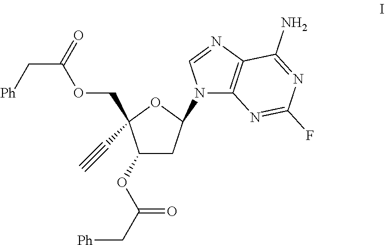 [SMILES: C#C[C@]1(COC(=O)Cc2ccccc2)O[C@@H](n2cnc3c(N)nc(F)nc32)C[C@@H]1OC(=O)Cc1ccccc1]

or a co-crystal, solvate, salt, or combination thereof;
comprising reacting a compound of formula XXII:

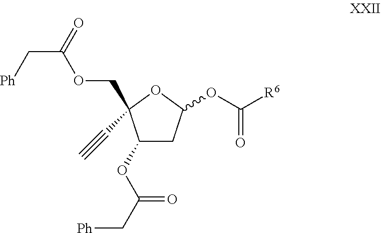

or a co-crystal, solvate, salt, or combination thereof,
wherein R⁶ is selected from C₁₋₄ alkyl, C₁₋₄ haloalkyl, C₁₋₄ alkoxy, benzyloxy, or C₆₋₁₀ aryl optionally substituted with 1 to 5 substituents independently selected from C₁₋₄ alkyl, C₁₋₄ haloalkyl, halo, C₁₋₄ alkoxy, or benzyloxy,
with a compound of formula XV:

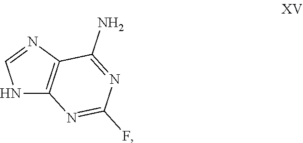 [SMILES: Nc1nc(F)nc2[nH]cnc12]

a silylating reagent, and
a Lewis acid,
   to provide the compound of formula I or a co-crystal, solvate, salt, or combination thereof.

The details of one or more embodiments of the invention are set forth in the accompanying description below. Other features, objects, and advantages of the invention will be apparent from the description and drawings, and from the claims.

### DETAILED DESCRIPTION

The present disclosure provides processes of preparing a compound of formula I:

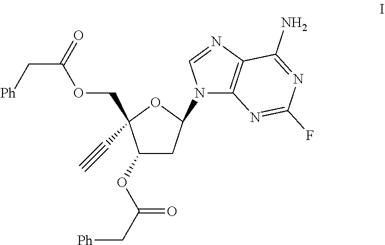 [SMILES: C#C[C@]1(COC(=O)Cc2ccccc2)O[C@@H](n2cnc3c(N)nc(F)nc32)C[C@@H]1OC(=O)Cc1ccccc1]

and co-crystals, solvates, salts, or combinations thereof. The compound of formula I is also known as (2R,3S,5R)-5-(6-amino-2-fluoro-9H-purin-9-yl)-2-ethynyl-2-((2-phenylacetoxy)methyl)tetrahydrofuran-3-yl 2-phenylacetate and is disclosed in U.S. Publication Nos. 20220323476A1 and 20220332751A1, the disclosures of each of which are incorporated herein by reference in their entireties.

The compound of formula I is a prodrug of the compound of formula XVII (i.e., islatravir, 4′-ethynyl-2-fluoro-2′-deoxyadenosine, or (2R,3S,5R)-5-(6-amino-2-fluoro-9H-purin-9-yl)-2-ethynyl-2-(hydroxymethyl)tetrahydrofuran-3-ol), a nucleoside reverse transcriptase translocation inhibitor (NRTTI) useful for treating a Retroviridae viral infection, including an infection caused by the HIV virus.

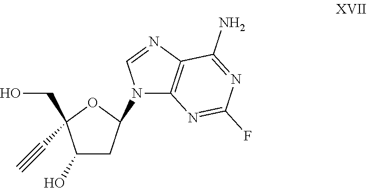 [SMILES: C#C[C@]1(CO)O[C@@H](n2cnc3c(N)nc(F)nc32)C[C@@H]1O]

The description below is made with the understanding that the present disclosure is to be considered as an exemplification of the claimed subject matter, and is not intended to limit the appended claims to the specific embodiments illustrated. The headings used throughout this disclosure are provided for convenience and are not to be construed to limit the claims in any way. Embodiments illustrated under any heading may be combined with embodiments illustrated under any other heading. Unless defined otherwise, all technical and scientific terms used herein have the same meaning as commonly understood by one of ordinary skill in the art.

When trade names are used herein, it is intended to independently include the tradename product and the active pharmaceutical ingredient(s) of the tradename product.

As used herein and in the appended claims, the singular forms “a” and “an”, and “the” include plural referents unless the context clearly dictates otherwise. Thus, e.g., reference to “the compound” includes a plurality of such compounds and reference to “the assay” includes reference to one or more assays, and so forth.

The absolute stereochemistry is specified according to the Cahn-Ingold-Prelog R—S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and/or hindered rotation about a bond axis and may thus give rise to enantiomers, diastereomers, and other stereoisomeric forms that may be defined, in terms of absolute stereochemistry, as (R)- or (S)-. The present disclosure is meant to include all such possible isomers, including racemic mixtures, scalemic mixtures, diastereomeric mixtures, optically pure forms and intermediate mixtures. Optically active (R)- and (S)-isomers may be prepared using chiral synthons or chiral reagents, or resolved using conventional techniques. All stereochemistry for compounds depicted herein is not intended to be limiting.

Except as expressly defined otherwise, the present disclosure includes all tautomers of compounds detailed herein, even if only one tautomer is expressly represented (e.g., both tautomeric forms are intended and described by the presentation of one tautomeric form where a pair of two tautomers may exist). For example, if reference is made to a compound containing an amide (e.g., by structure or chemical name), it is understood that the corresponding imidic acid tautomer is included by this disclosure and described the same as if the amide were expressly recited either alone or together with the imidic acid. Where more than two tautomers may exist, the present disclosure includes all such tautomers even if only a single tautomeric form is depicted by chemical name and/or structure.

Compounds described herein may have chiral centers and/or geometric isomeric centers (E- and Z-isomers), and it is to be understood that all such optical, enantiomeric, diastereoisomeric and geometric isomers are encompassed. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s). As used herein, “scalemic mixture” is a mixture of stereoisomers at a ratio other than 1:1.

Also provided are pharmaceutically acceptable hydrates, solvates, co-crystals, tautomeric forms, polymorphs, and prodrugs of the compounds described herein.

The term “hydrate” refers to the complex formed by the combining of a compound of Formula I, or any Formula disclosed herein, and water.

The term “solvate” refers to a complex formed by the combining of a compound of Formula I, or any other Formula as disclosed herein, and a solvent or a crystalline solid containing amounts of a solvent incorporated within the crystal structure. As used herein, the term “solvate” includes hydrates.

The term “co-crystal” refers to a crystalline material formed by combining a compound of Formula I, or any Formula disclosed herein and one or more co-crystal formers (i.e., a molecule, ion or atom). In certain instances, co-crystals may have improved properties as compared to the parent form (i.e., the free molecule, zwitterion, etc.) or a salt of the parent compound. Improved properties can be increased solubility, increased dissolution, increased bioavailability, increased dose response, decreased hygroscopicity, a crystalline form of a normally amorphous compound, a crystalline form of a difficult to salt or unsaltable compound, decreased form diversity, more desired morphology, and the like. Methods for making and characterizing co-crystals are known to those of skill in the art.

Any formula or structure given herein, including Formula I, or any Formula disclosed herein, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to 2H (deuterium, D), 3H (tritium), 11C, 13C, 14C, 15N, 18F, 31P, 32P, 35S, 36Cl and 125I. Various isotopically labeled compounds of the present disclosure, for example those into which radioactive isotopes such as 3H, 13C and 14C are incorporated. Such isotopically labeled compounds may be useful in metabolic studies, reaction kinetic studies. detection or imaging techniques, such as positron emission tomography (PET) or single-photon emission computed tomography (SPECT) including drug or substrate tissue distribution assays or in radioactive treatment of patients.

The disclosure also includes compounds of Formula I, or any Formula disclosed herein, in which from 1 to “n” hydrogens attached to a carbon atom is/are replaced by deuterium, in which n is the number of hydrogens in the molecule. Such compounds exhibit increased resistance to metabolism and are thus useful for increasing the half-life of any compound of Formula I when administered to a mammal. See, for example, Foster, “Deuterium Isotope Effects in Studies of Drug Metabolism”, Trends Pharmacol. Sci. 5(12):524-527 (1984). Such compounds are synthesized by means well known in the art, for example by employing starting materials in which one or more hydrogen atoms have been replaced by deuterium.

Deuterium labeled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life or reduced dosage requirements. An 18F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. Further, substitution with heavier isotopes, particularly deuterium (i.e., 2H or D) may afford certain therapeutic advantages resulting from greater metabolic stability, for example increased in vivo half-life or reduced dosage requirements or an improvement in therapeutic index. It is understood that deuterium in this context is regarded as a substituent in the compound of the Formula I, or any Formula disclosed herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

The modifier “about” used in connection with a quantity is inclusive of the stated value and has the meaning dictated by the context (e.g., includes the degree of error associated with measurement of the particular quantity).

“Alkyl” is a straight or branched saturated hydrocarbon. For example, an alkyl group can have 1 to 8 carbon atoms (i.e., (C₁-C₈)alkyl) or 1 to 6 carbon atoms (i.e., (C₁-C₆ alkyl) or 1 to 4 carbon atoms (i.e., (C₁-C₄)alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃) 2), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH CH₂CH₂CH₃), 2-pentyl (—CH(CH₃)CH₂CH₂CH₃), 3-pentyl (—CH(CH₂CH₃)₂), 2-methyl-2-butyl (—C (CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, and octyl (—(CH₂)₇CH₃).

The term “alkoxy” as used herein refers to a group of formula —O-alkyl. Example alkoxy groups include, but are not limited to, methoxy, ethoxy, propoxy (e.g., n-propoxy and isopropoxy), and t-butoxy.

The term “halo” as used herein refers to fluoro, chloro, bromo and iodo.

The term “haloalkyl” as used herein refers to an alkyl as defined herein, wherein one or more hydrogen atoms of the alkyl are each independently replaced by a halo substituent. For example, (C₁-C₆)haloalkyl is a (C₁-C₆)alkyl wherein one or more of the hydrogen atoms of the (C₁-C₆)alkyl have been replaced by a halo substituent. Examples of haloalkyls include but are not limited to fluoromethyl, fluorochloromethyl, difluoromethyl, difluorochloromethyl, trifluoromethyl, 1,1,1, trifluoroethyl and pentafluoroethyl.

The term “aryl” as used herein refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, 6 to 12 carbon atoms, or 6 to 10 carbon atoms. Aryl includes a phenyl radical. Aryl also includes multiple condensed ring systems (e.g., ring systems comprising 2, 3 or 4 rings) having about 9 to 20 carbon atoms in which at least one ring is aromatic and wherein the other rings may be aromatic or not aromatic (i.e., carbocycle). Such multiple condensed ring systems are optionally substituted with one or more (e.g., 1, 2 or 3) oxo groups on any carbocycle portion of the multiple condensed ring system. The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring. It is also to be understood that when reference is made to a certain atom-range membered aryl (e.g., 6-10 membered aryl), the atom range is for the total ring atoms of the aryl. For example, a 6-membered aryl would include phenyl and a 10-membered aryl would include naphthyl and 1, 2, 3, 4-tetrahydronaphthyl. Non-limiting examples of aryl groups include, but are not limited to, phenyl (Ph), indenyl, naphthyl, 1,2,3,4-tetrahydronaphthyl, anthracenyl, and the like.

The term “cycloalkyl” refers to a cyclic alkyl and alkenyl groups. A cycloalkyl group can have one or more cyclic rings and includes fused and bridged groups that are fully saturated or partially unsaturated. Examples include, but are not limited to, cyclopropyl, cyclobutyl, cyclopentyl, cyclohexyl, adamantyl, methylcycloproyl (cyclopropylmethyl), ethylcyclopropyl, cyclohexenyl and the like.

Except as otherwise noted, the methods and techniques of the present disclosure are generally performed according to conventional methods well known in the art and as described in various general and more specific references that are cited and discussed throughout the present specification. See, e.g., Loudon, Organic Chemistry, 5^{th} edition, New York: Oxford University Press, 2009; Smith, March's Advanced Organic Chemistry: Reactions, Mechanisms, and Structure, 7^{th} edition. Wiley-Interscience, 2013.

In certain instances, the processes disclosed herein involve a step of forming a salt of a compound of the present disclosure.

Compounds as described herein can be purified by any of the means known in the art, including chromatographic means, such as high performance liquid chromatography (HPLC), preparative thin layer chromatography, flash column chromatography, supercritical fluid chromatography (SFC), and ion exchange chromatography. Any suitable stationary phase can be used, including normal and reversed phases as well as ionic resins. Most typically the disclosed compounds are purified via silica gel and/or alumina chromatography. See, e.g., Introduction to Modern Liquid Chromatography, 2^{nd} ed., ed. L. R. Snyder and J. J. Kirkland, John Wiley and Sons, 1979; and Thin Layer Chromatography, E. Stahl (ed.), Springer-Verlag, New York, 1969.

During any of the processes for preparation of the subject compounds, it may be necessary and/or desirable to protect sensitive or reactive groups on any of the molecules concerned. This may be achieved by means of conventional protecting groups as described in standard works, such as T. W. Greene and P. G. M. Wuts, Protective Groups in Organic Synthesis, 4^{th} ed., Wiley, New York 2006. The protecting groups may be removed at a convenient subsequent stage using methods known from the art.

Exemplary chemical entities useful in methods of the embodiments will now be described by reference to illustrative synthetic schemes for their general preparation herein and the specific examples that follow. One of skill in the art will recognize that the transformations shown in the schemes below may be performed in any order that is compatible with the functionality of the particular pendant groups. In some embodiments, each of the reactions depicted in the general schemes is run at a temperature from about −80° C. to the reflux temperature of the organic solvent used.

In some embodiments, provided herein is a process of preparing a compound of formula I, or co-crystal, solvate, salt, or combination thereof; comprising reacting compound of formula XVII or a co-crystal, solvate, salt, or combination thereof, with a compound of formula XVIII:

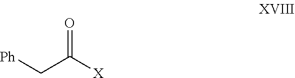

wherein X is selected from halo, —OCOCH₂Ph, and OH, and
a base,
   to provide the compound of formula I or a co-crystal, solvate, salt, or combination thereof.

In some embodiments, X is halo. In some embodiments, X is Cl or Br. In some embodiments, X is Cl.

In some embodiments, X is OH, and the reacting is performed in the presence of an activating reagent. In some embodiments, the activating reagent comprises a chlorinating reagent, 1,1-carbonyldiimidazole, a carbodiimide, a peptide coupling reagent, or combination thereof. In some embodiments, the chlorinating reagent comprises oxalyl chloride, thionyl chloride, phosphoryl chloride, or a combination thereof. In some embodiments, the carbodiimide is 1-ethyl-3-(3-dimethylaminopropyl)carbodiimide. In some embodiments, the peptide coupling reagent comprises HATU, propylphosphonic anhydride, isobutyl chloroformate, or a combination thereof. In some embodiments, the activating reagent is selected from the group consisting of oxalyl chloride, thionyl chloride, phosphoryl chloride, 1,1-carbonyldiimidazole, 1-ethyl-3-(3-dimethylaminopropyl)carbodiimide, HATU, propylphosphonic anhydride, and isobutyl chloroformate.

In some embodiments, the base comprises an aromatic amine, tertiary alkyl amine, carbonate, or combination thereof. In some embodiments, the base comprises an aromatic amine. In some embodiments, the aromatic amine comprises pyridine, 2,6-lutidine, pyridazine, imidazole, pyrimidine, pyrazine, or a combination thereof. In some embodiments, the base is N-methylimidazole. In some embodiments, the tertiary alkyl amine comprises triethylamine, diisopropylethylamine, N-methylmorpholine, or a combination thereof. In some embodiments, the carbonate is an alkali metal carbonate (e.g., lithium carbonate, sodium carbonate, cesium carbonate).

In some embodiments, the reacting is performed in a solvent (e.g. a first solvent) comprising a polar aprotic solvent (e.g., acetonitrile, N,N-dimethylformamide, N,N-dimethylacetamide, N-methyl-2-pyrrolidone), ether (e.g., tetrahydrofuran, 2-methyltetrahydrofuran, tert-butyl methyl ether, 1,2-dimethoxyethane, 1,4-dioxane, diglyme, dimethyl isosorbide), ketone (e.g., acetone, methyl ethyl ketone, methyl isobutyl ketone), halogenated solvent (e.g., 1,2-dichloroethane, chloroform, chlorobenzene, dichloromethane, α,α,α-trifluorotoluene), or a combination thereof. In some embodiments, the reacting is performed in a solvent (e.g., a first solvent) comprising acetone and acetonitrile.

In some embodiments, the reacting is carried out in a temperature range of from about −20° C. to about 10° C. In some embodiments, the reacting is carried out in a temperature range of from about −20° C. to about 0° C., such as about −10° C.

In some embodiments, the reacting is performed in the presence of a catalyst. In some embodiments, the catalyst is selected from the group consisting of 4-dimethylaminopyridine, imidazole, triphenylphosphine oxide, and 1-hydroxy-7-azabenzotriazole.

In some embodiments, the process further comprises preparing the compound of formula XVII or a co-crystal, solvate, salt, or combination thereof by a process comprising: reacting a compound of formula XVI-A:

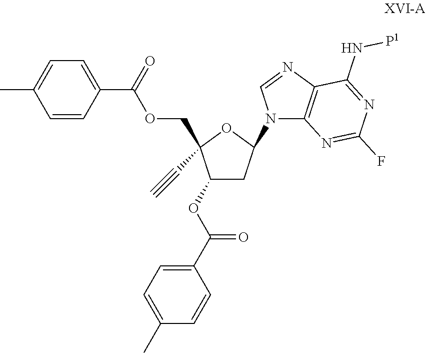

or a co-crystal, solvate, salt, or combination thereof,
wherein P¹ is H or (R⁴)₃Si, wherein each R⁴ is independently C₁₋₆ alkyl, with a de-acylation reagent, and
   a second base,
      to provide the compound of formula XVII or a co-crystal, solvate, salt, or combination thereof.

In some embodiments, each R⁴ is independently C₁₋₃ alkyl. In some embodiments, P¹ is trimethylsilyl or triisopropylsiyl. In some embodiments, P¹ is trimethylsilyl. In some embodiments, P¹ is H.

In some embodiments, the compound of formula XVI-A or a co-crystal, solvate, salt, or combination thereof is a compound of formula XVI:

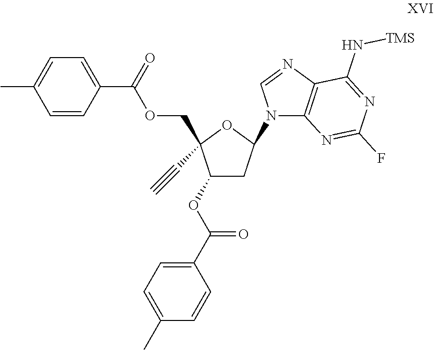 [SMILES: C#C[C@]1(COC(=O)c2ccc(C)cc2)O[C@@H](n2cnc3c(N[Si](C)(C)C)nc(F)nc32)C[C@@H]1OC(=O)c1ccc(C)cc1]

or a co-crystal, solvate, salt, or combination thereof.

In some embodiments, the de-acylation reagent is a C₁₋₄ alcohol. In some embodiments, the de-acylation reagent comprises methanol, ethanol, 1-propanol, 2-propanol, or another nucleophilic alcohol (e.g., tert-butanol), or a combination thereof. In some embodiments, the de-acylation reagent is methanol.

In some embodiments, the second base comprises a hydroxide, oxide, alkoxide, carbonate, or combination thereof. For example, the second base is selected from the group consisting of an alkali metal hydroxide (e.g., sodium hydroxide, potassium hydroxide, lithium hydroxide), an alkali metal oxide (e.g., sodium oxide), an alkaline earth metal oxide (e.g., calcium oxide, magnesium oxide), an alkali metal alkoxide (e.g., sodium methoxide, potassium methoxide, lithium methoxide, potassium tert-butoxide, sodium tert-butoxide), an alkali metal carbonate (e.g., sodium carbonate, cesium carbonate, potassium carbonate), or a combination thereof. In some embodiments, the second base comprises an alkali metal alkoxide. In some embodiments, the second base is sodium methoxide.

In some embodiments, from about 0.02 to about 0.2 equivalents of the second base are utilized relative to the compound of formula XVI-A or a co-crystal, solvate, salt, or combination thereof. In some embodiments, from about 0.02 to about 0.15 equivalents, about 0.02 to about 0.10 equivalents, or about 0.02 to about 0.05 equivalents of the second base are utilized relative to the compound of formula XVI-A or a co-crystal, solvate, salt, or combination thereof. In some embodiments, about 0.03 equivalents of the second base are utilized relative to the compound of formula XVI-A or a co-crystal, solvate, salt, or combination thereof.

In some embodiments, the reacting the compound of formula XVI-A or a co-crystal, solvate, salt, or combination thereof is performed in a solvent (e.g. a second solvent) comprising a polar aprotic solvent (e.g., N,N-dimethylformamide, N,N-dimethylacetamide, N-methyl-2-pyrrolidone, acetonitrile), ether (e.g., tetrahydrofuran, 2-methyltetrahydrofuran, 1,2-dimethoxyethane, tert-butyl methyl ether, 1,4-dioxane, diglyme, dimethyl isosorbide), ketone (e.g., acetone, methyl ethyl ketone, methyl isobutyl ketone), halogenated solvent (e.g., 1,2-dichloroethane, chloroform, chlorobenzene, dichloromethane, α,α,α-trifluorotoluene), or a combination thereof. In some embodiments, the reacting the compound of formula XVI-A or a co-crystal, solvate, salt, or combination thereof is performed in a solvent (e.g., a second solvent) comprising tetrahydrofuran and methanol.

In some embodiments, the reacting the compound of formula XVI-A or a co-crystal, solvate, salt, or combination thereof is carried out in a temperature range of from about −20° C. to about 20° C. In some embodiments, the reacting the compound of formula XVI-A or a co-crystal, solvate, salt, or combination thereof is carried out in a temperature range of from about −10° C. to about 5° C., such as about 0° C.

In some embodiments, the process further comprises preparing the compound of formula XVI-A or a co-crystal, solvate, salt, or combination thereof by a process comprising reacting a compound of formula XIV:

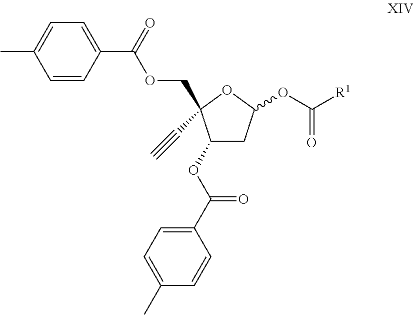

or a co-crystal, solvate, salt, or combination thereof,
wherein R¹ is selected from C₁₋₄ alkyl, C₁₋₄ haloalkyl, C₁₋₄ alkoxy, benzyloxy, or C₆₋₁₀ aryl optionally substituted with 1 to 5 substituents independently selected from C₁₋₄ alkyl, C₁₋₄ haloalkyl, halo, C₁₋₄ alkoxy, or benzyloxy,
with a compound of formula XV:

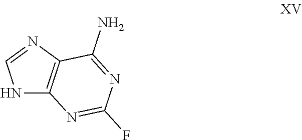 [SMILES: Nc1nc(F)nc2[nH]cnc12]

or a co-crystal, solvate, salt, or combination thereof,
a silylating reagent, and
a Lewis acid,
to provide the compound of formula XVI-A or a co-crystal, solvate, salt, or combination thereof.

In some embodiments, R¹ is C₁₋₄ alkyl. In some embodiments, R¹ is methyl. In some embodiments, R¹ is ethyl. In some embodiments, R¹ is C₁₋₄ haloalkyl. In some embodiments, R¹ is trifluoromethyl.

In some embodiments, R¹ is C₆₋₁₀ aryl optionally substituted with 1 to 5 substituents independently selected from C₁₋₄ alkyl, C₁₋₄ haloalkyl, halo, C₁₋₄ alkoxy, or benzyloxy. In some embodiments, R¹ is phenyl. In some embodiments, R¹ is tolyl.

In some embodiments, the Lewis acid is selected from the group consisting of trimethylsilyl trifluoromethanesulfonate, boron trifluoride diethyl etherate, and tin tetrachloride. In some embodiments, the Lewis acid is trimethylsilyl trifluoromethanesulfonate.

In some embodiments, the silylating reagent is selected from the group consisting of bis(trialkylsilyl)acetamide and bis(trialkylsilyl)trifluoroacetamide. In some embodiments, the silylating reagent is selected from the group consisting of bis(trimethylsilyl)acetamide and bis(trimethylsilyl)trifluoroacetamide. In some embodiments, the silylating reagent is bis(trimethylsilyl)acetamide.

In some embodiments, the reacting the compound of formula XIV or a co-crystal, solvate, salt, or combination thereof is performed in a solvent (e.g. a third solvent) comprising a polar aprotic solvent (e.g., acetonitrile, propionitrile, benzonitrile), halogenated solvent (e.g., dichloromethane, 1,2-dichloroethane, chloroform, chlorobenzene, α,α,α-trifluorotoluene), toluene, xylene, anisole, trifluorotoluene, or a combination thereof. In some embodiments, the reacting the compound of formula XIV or a co-crystal, solvate, salt, or combination thereof is performed in a solvent (e.g., a third solvent) comprising acetonitrile.

In some embodiments, the reacting the compound of formula XIV or a co-crystal, solvate, salt, or combination thereof is carried out in a temperature range of from about 60° C. to about 100° C. In some embodiments, the reacting the compound of formula XIV or a co-crystal, solvate, salt, or combination thereof is carried out in a temperature range of from about 70° C. to about 80° C., such as about 75° C.

In some embodiments, the process further comprises preparing the compound of formula XIV or a co-crystal, solvate, salt, or combination thereof by a process comprising: reacting a compound of formula XII:

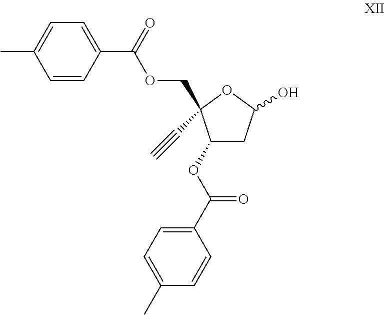 [SMILES: C#C[C@]1(COC(=O)c2ccc(C)cc2)OC(O)C[C@@H]1OC(=O)c1ccc(C)cc1]

or a co-crystal, solvate, salt, or combination thereof, with
a compound selected from the group consisting of a compound of formula XIII-A:

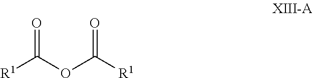

and a compound of formula XIII-B:

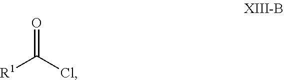

a third base, and
a second catalyst,
to provide the compound of formula XIV or a co-crystal, solvate, salt, or combination thereof.

In some embodiments, the process comprises reacting the compound of formula XII or a co-crystal, solvate, salt, or combination thereof with the compound of formula XIII-A. In some embodiments, the compound of formula XIII-A is selected from the group consisting of acetic anhydride, trifluoroacetic anhydride, propionic anhydride, benzoic anhydride, toluic anhydride, and di-tert-butyl decarbonate (Boc-anhydride). In some embodiments, the compound of formula XIII-A is acetic anhydride.

In some embodiments, R¹ of formula XIII-A is selected from C₁₋₄ alkyl, C₁₋₄ haloalkyl, C₁₋₄ alkoxy, benzyloxy, or C₆₋₁₀ aryl optionally substituted with 1 to 5 substituents independently selected from C₁₋₄ alkyl, C₁₋₄ haloalkyl, halo, C₁₋₄ alkoxy, or benzyloxy. In some embodiments, R¹ of formula XIII-A is C₁₋₄ alkyl. In some embodiments, R¹ of formula XIII-A is methyl. In some embodiments, R¹ of formula XIII-A is ethyl. In some embodiments, R¹ of formula XIII-A is C₁₋₄ haloalkyl. In some embodiments, R¹ of formula XIII-A is trifluoromethyl. In some embodiments, R¹ of formula XIII-A is C₆₋₁₀ aryl optionally substituted with 1 to 5 substituents independently selected from C₁₋₄ alkyl, C₁₋₄ haloalkyl, halo, C₁₋₄ alkoxy, or benzyloxy. In some embodiments, R¹ of formula XIII-A is phenyl. In some embodiments, R¹ of formula XIII-A is tolyl.

In some embodiments, the process comprises reacting the compound of formula XII or a co-crystal, solvate, salt, or combination thereof with the compound of formula XIII-B. In some embodiments, the compound of XIII-B is selected from the group consisting of acetyl chloride, propionyl chloride, benzoyl chloride, toluoyl chloride, methyl chloroformate, ethyl chloroformate, and benzyl chloroformate.

In some embodiments, R¹ of formula XIII-B is selected from C₁₋₄ alkyl, C₁₋₄ haloalkyl, C₁₋₄ alkoxy, benzyloxy, or C₆₋₁₀ aryl optionally substituted with 1 to 5 substituents independently selected from C₁₋₄ alkyl, C₁₋₄ haloalkyl, halo, C₁₋₄ alkoxy, or benzyloxy. In some embodiments, R¹ of formula XIII-B is C₁₋₄ alkyl. In some embodiments, R¹ of formula XIII-B is methyl. In some embodiments, R¹ of formula XIII-B is ethyl In some embodiments, R¹ of formula XIII-B is C₁₋₄ haloalkyl. In some embodiments, R¹ of formula XIII-B is trifluoromethyl. In some embodiments, R¹ of formula XIII-B is C₆₋₁₀ aryl optionally substituted with 1 to 5 substituents independently selected from C₁₋₄ alkyl, C₁₋₄ haloalkyl, halo, C₁₋₄ alkoxy, or benzyloxy. In some embodiments, R¹ of formula XIII-B is phenyl. In some embodiments, R¹ of formula XIII-B is tolyl.

In some embodiments, the third base comprises an aromatic amine, tertiary alkyl amine, carbonate, or combination thereof. In some embodiments, the aromatic amine comprises pyridine, 2,6-lutidine, pyridazine, imidazole, pyrimidine, pyrazine, or a combination thereof. In some embodiments, the tertiary alkyl amine comprises triethylamine, diisopropylethy lamines, N-methylmorpholine, or a combination thereof. In some embodiments, the carbonate comprises an alkali metal carbonate (e.g., lithium carbonate, sodium carbonate, cesium carbonate).

In some embodiments, the third base comprises a tertiary alkyl amine. In some embodiments, the third base is triethylamine.

In some embodiments, the second catalyst is selected from the group consisting of 4-dimethylaminopyridine, imidazole, triphenylphosphine oxide, and 1-hydroxy-7-azabenzotriazole. In some embodiments, the second catalyst is 4-dimethylaminopyridine.

In some embodiments, the reacting the compound of formula XII or a a co-crystal, solvate, salt, or combination thereof is performed in a solvent (e.g. a fourth solvent) comprising a hydrocarbon (e.g. toluene, xylene), ether (e.g. tetrahydrofuran, 2-methyltetrahydrofuran, tert-butyl methyl ether, 1,2-dimethoxyethane, 1,4-dioxane, diglyme, dimethyl isosorbide), ketone (e.g. acetone, methyl ethyl ketone, methyl isobutyl ketone), halogenated solvent (e.g., 1,2-dichloroethane, chloroform, chlorobenzene, dichloromethane, α,α,α-trifluorotoluene), polar aprotic solvent (e.g., N,N-dimethylformamide, N,N-dimethylacetamide, N-methyl-2-pyrrolidone, acetonitrile), ester (e.g., ethyl acetate, isopropyl acetate), or a combination thereof. In some embodiments, the reacting the compound of formula XII or a co-crystal, solvate, salt, or combination thereof is performed in a solvent (e.g., a fourth solvent) comprising toluene.

In some embodiments, the reacting the compound of formula XII or a co-crystal, solvate, salt, or combination thereof is carried out in a temperature range of from about −50° C. to about 50° C. In some embodiments, the reacting the compound of formula XII or a co-crystal, solvate, salt, or combination thereof is carried out in a temperature range of from about −5° C. to about 15° C., such as about 5° C.

In some embodiments, the process further comprises preparing the compound of formula XII or a co-crystal, solvate, salt, or combination thereof by a process comprising: reducing a compound of formula XI:

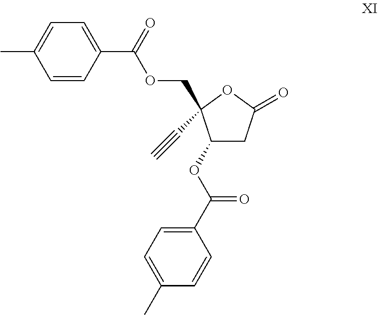 [SMILES: C#C[C@]1(COC(=O)c2ccc(C)cc2)OC(=O)C[C@@H]1OC(=O)c1ccc(C)cc1]

or a co-crystal, solvate, salt, or combination thereof, with
a reducing agent,
to provide the compound of formula XII or a co-crystal, solvate, salt, or combination thereof.

In some embodiments, the reducing agent is selected from the group consisting of diisobutylaluminum hydride, sodium bis(2-methoxyethoxy)aluminum hydride, lithium aluminum hydride, lithium borohydride, and lithium tri-t-butoxyaluminum hydride. In some embodiments, the reducing agent is diisobutylaluminum hydride.

In some embodiments, the reducing is performed in a solvent (e.g., a fifth solvent) comprising an ether (e.g., tetrahydrofuran, 2-methyltetrahydrofuran, tert-butyl methyl ether, 1,4-dioxane, 1,2-dimethoxyethane, diglyme, dimethyl isosorbide), a hydrocarbon (e.g., toluene, xylene), or a combination thereof. In some embodiments, the reducing is performed in a solvent (e.g., a fifth solvent) comprising 1,2-dimethoxyethane.

In some embodiments, the reducing is carried out in a temperature range of from about −80° C. to about −30° C. In some embodiments, the reducing is carried out in a temperature range of from about −78° C. to about −30° C., or about −60° C. to about −40° C., such as about −50° C.

In some embodiments, the process further comprises preparing the compound of formula XI or a co-crystal, solvate, salt, or combination thereof by a process comprising:
esterifying a compound of formula X:

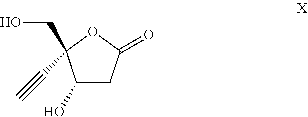

or a co-crystal, solvate, salt, or combination thereof, with
a fourth base,
a third catalyst, and
an acylating reagent,
to provide the compound of formula XI or a co-crystal, solvate, salt, or combination thereof.

In some embodiments, the fourth base is an organic base or an inorganic base.

In some embodiments, the organic base is selected from the group consisting of N-methylimidazole, triethylamine, Hunig's base, pyridine, imidazole, DABCO, and DBU. In some embodiments, the fourth base is N-methylimidazole.

In some embodiments, the inorganic base comprises an alkali metal carbonate, alkaline earth metal carbonate, or alkali metal hydroxide. In some embodiments, the inorganic base is selected from the group consisting of Na₂CO₃, Cs₂CO₃, K₂CO₃, NaOH, and KOH.

In some embodiments, the third catalyst comprises an aromatic base. In some embodiments, the third catalyst is selected from the group consisting of 4-dimethylaminopyridine, pyridine, imidazole, and 4-pyrrolidinylpyridine. In some embodiments, the third catalyst is 4-dimethylaminopyridine.

In some embodiments, the acylating reagent is selected from the group consisting of toluoyl halide and toluic acid. In some embodiments, the acylating reagent is selected from the group consisting of toluoyl chloride and toluic acid. In some embodiments, the acylating reagent is toluoyl chloride.

In some embodiments, the acylating reagent is toluic acid, and the esterifying is performed in the presence of a carboxylic acid activator. In some embodiments, the carboxylic acid activator is selected from the group consisting of 1,1′-carbonyldiimidazole, thionyl chloride, and oxalyl chloride.

In some embodiments, the esterifying is performed in a solvent (e.g., a sixth solvent) comprising a polar aprotic solvent. For example, the polar aprotic solvent comprises N,N-dimethylformamide, N,N-dimethylacetamide, N-methyl-2-pyrrolidone, ethyl acetate, THF, 1,2-dimethoxyethane, acetonitrile, dichloromethane, or a combination thereof. In some embodiments, the esterifying is performed in a solvent (e.g., a sixth solvent) comprising 1,2-dimethoxyethane and acetonitrile.

In some embodiments, the esterifying is carried out in a temperature range of from about −20° C. to about 40° C. In some embodiments, the esterifying is carried out in a temperature range of from about −20° C. to about 0° C.

In some embodiments, provided herein is a process for preparing a compound of formula I:

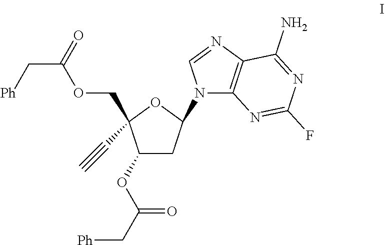 [SMILES: C#C[C@]1(COC(=O)Cc2ccccc2)O[C@@H](n2cnc3c(N)nc(F)nc32)C[C@@H]1OC(=O)Cc1ccccc1]

or a co-crystal, solvate, salt, or combination thereof;
comprising reacting a compound of formula XXII:

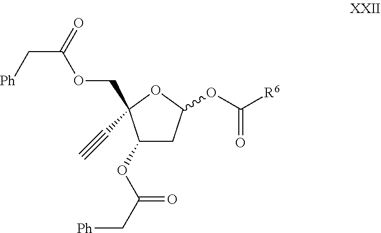

or a co-crystal, solvate, salt, or combination thereof,
wherein R⁶ is selected from C₁₋₄ alkyl, C₁₋₄ haloalkyl, C₁₋₄ alkoxy, benzyloxy, or C₆₋₁₀ aryl optionally substituted with 1 to 5 substituents independently selected from C₁₋₄ alkyl, C₁₋₄ haloalkyl, halo, C₁₋₄ alkoxy, or benzyloxy,
with a compound of formula XV:

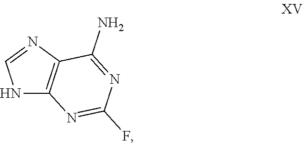 [SMILES: Nc1nc(F)nc2[nH]cnc12]

a silylating reagent, and
a Lewis acid,
   to provide the compound of formula I or a co-crystal, solvate, salt, or combination thereof.

In some embodiments, R⁶ is C₁₋₄ alkyl. In some embodiments, R⁶ is methyl. In some embodiments, R⁶ is ethyl. In some embodiments, R⁶ is C₁₋₄ haloalkyl. In some embodiments, R⁶ is trifluoromethyl.

In some embodiments, R⁶ is C₆₋₁₀ aryl optionally substituted with 1 to 5 substituents independently selected from C₁₋₄ alkyl, C₁₋₄ haloalkyl, halo, C₁₋₄ alkoxy, or benzyloxy. In some embodiments, R⁶ is phenyl. In some embodiments, R⁶ is tolyl.

In some embodiments, the Lewis acid is selected from the group consisting of trimethylsilyl trifluoromethanesulfonate, boron trifluoride diethyl etherate, and tin tetrachloride. In some embodiments, the Lewis acid is trimethylsilyl trifluoromethanesulfonate.

In some embodiments, the silylating reagent is selected from the group consisting of bis(trialkylsilyl)acetamide and bis(trialkylsilyl)trifluoroacetamide. In some embodiments, the silylating reagent is selected from the group consisting of bis(trimethylsilyl)acetamide and bis(trimethylsilyl)trifluoroacetamide. In some embodiments, the silylating reagent is bis(trimethylsilyl)acetamide.

In some embodiments, the reacting is performed in a solvent (e.g., a first solvent) comprising a polar aprotic solvent (e.g., acetonitrile, propionitrile, benzonitrile), halogenated solvent (e.g., dichloromethane, 1,2-dichloroethane, chloroform, chlorobenzene, α,α,α-trifluorotoluene), toluene, xylene, anisole, trifluorotoluene, or a combination thereof. In some embodiments, the reacting is performed in a solvent (e.g., a first solvent) comprising acetonitrile.

In some embodiments, the reacting is carried out in a temperature range of from about 60° C. to about 100° C. In some embodiments, the reacting is carried out in a temperature range of from about 70° C. to about 80° C., such as about 75° C.

In some embodiments, the process further comprises preparing the compound of formula XXII or a co-crystal, solvate, salt, or combination thereof by a process comprising:
reacting a compound of formula XX:

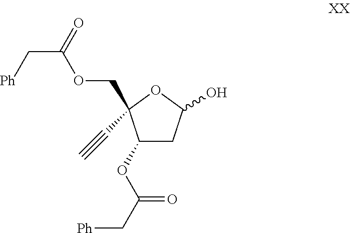 [SMILES: C#C[C@]1(COC(=O)Cc2ccccc2)OC(O)C[C@@H]1OC(=O)Cc1ccccc1]

or a co-crystal, solvate, salt, or combination thereof, with
a compound selected from the group consisting of a compound of formula XXI-A:

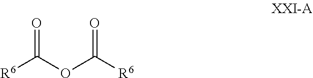

and a compound of formula XXI-B:

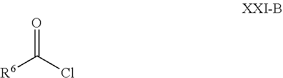

a base, and
a catalyst,
   to provide the compound of formula XXII or a co-crystal, solvate, salt, or combination thereof.

In some embodiments, the process comprises reacting the compound of formula XX or a co-crystal, solvate, salt, or combination thereof with the compound of formula XXI-A. In some embodiments, the compound of formula XXI-A is selected from the group consisting of acetic anhydride, trifluoroacetic anhydride, propionic anhydride, benzoic anhydride, toluic anhydride, and di-tert-butyl decarbonate (Boc-anhydride). In some embodiments, the compound of formula XXI-A is acetic anhydride.

In some embodiments, R⁶ of formula XXI-A is selected from C₁₋₄ alkyl, C₁₋₄ haloalkyl, C₁₋₄ alkoxy, benzyloxy, or C₆₋₁₀ aryl optionally substituted with 1 to 5 substituents independently selected from C₁₋₄ alkyl, C₁₋₄ haloalkyl, halo, C₁₋₄ alkoxy, or benzyloxy. In some embodiments, R⁶ of formula XXI-A is C₁₋₄ alkyl. In some embodiments, R⁶ of formula XXI-A is methyl. In some embodiments, R⁶ of formula XXI-A is ethyl. In some embodiments, R⁶ of formula XXI-A is C₁₋₄ haloalkyl. In some embodiments, R⁶ of formula XXI-A is trifluoromethyl. In some embodiments, R⁶ of formula XXI-A is C₆₋₁₀ aryl optionally substituted with 1 to 5 substituents independently selected from C₁₋₄ alkyl, C₁₋₄ haloalkyl, halo, C₁₋₄ alkoxy, or benzyloxy. In some embodiments, R⁶ of formula XXI-A is phenyl. In some embodiments, R⁶ of formula XXI-A is tolyl.

In some embodiments, the process comprises reacting the compound of formula XX or a co-crystal, solvate, salt, or combination thereof with the compound of formula XXI-B. In some embodiments, the compound of XXI-B is selected from the group consisting of acetyl chloride, propionyl chloride, benzoyl chloride, toluoyl chloride, methyl chloroformate, ethyl chloroformate, and benzyl chloroformate.

In some embodiments, R⁶ of formula XXI-B is selected from C₁₋₄ alkyl, C₁₋₄ haloalkyl, C₁₋₄ alkoxy, benzyloxy, or C₆₋₁₀ aryl optionally substituted with 1 to 5 substituents independently selected from C₁₋₄ alkyl, C₁₋₄ haloalkyl, halo, C₁₋₄ alkoxy, or benzyloxy. In some embodiments, R⁶ of formula XXI-B is C₁₋₄ alkyl. In some embodiments, R⁶ of formula XXI-B is methyl. In some embodiments, R⁶ of formula XXI-B is ethyl. In some embodiments, R⁶ of formula XXI-B is C₁₋₄ haloalkyl. In some embodiments, R⁶ of formula XXI-B is trifluoromethyl. In some embodiments, R⁶ of formula XXI-B is C₆₋₁₀ aryl optionally substituted with 1 to 5 substituents independently selected from C₁₋₄ alkyl, C₁₋₄ haloalkyl, halo, C₁₋₄ alkoxy, or benzyloxy. In some embodiments, R⁶ of formula XXI-B is phenyl. In some embodiments, R⁶ of formula XXI-B is tolyl.

In some embodiments, the base comprises an aromatic amine, tertiary alkyl amine, carbonate, or combination thereof. In some embodiments, the aromatic amine comprises pyridine, 2,6-lutidine, pyridazine, imidazole, pyrimidine, pyrazine, or a combination thereof. In some embodiments, the tertiary alkyl amine comprises triethylamine, diisopropylethylamines, N-methylmorpholine, or a combination thereof. In some embodiments, the carbonate comprises an alkali metal carbonate (e.g., lithium carbonate, sodium carbonate, cesium carbonate).

In some embodiments, the base comprises a tertiary alkyl amine. In some embodiments, the base is triethylamine.

In some embodiments, the catalyst is selected from the group consisting of 4-dimethylaminopyridine, imidazole, triphenylphosphine oxide, and 1-hydroxy-7-azabenzotriazole. In some embodiments, the catalyst is 4-dimethylaminopyridine.

In some embodiments, the reacting the compound of formula XX or a co-crystal, solvate, salt, or combination thereof is performed in a solvent (e.g. a second solvent) comprising a hydrocarbon (e.g. toluene, xylene), ether (e.g. tetrahydrofuran, 2-methyltetrahydrofuran, tert-butyl methyl ether, 1,2-dimethoxyethane, 1,4-dioxane, diglyme, dimethyl isosorbide), ketone (e.g. acetone, methyl ethyl ketone, methyl isobutyl ketone), halogenated solvent (e.g., 1,2-dichloroethane, chloroform, chlorobenzene, dichloromethane, α,α,α-trifluorotoluene), polar aprotic solvent (e.g., N,N-dimethylformamide, N,N-dimethylacetamide, N-methyl-2-pyrrolidone, acetonitrile), ester (e.g., ethyl acetate, isopropyl acetate), or a combination thereof. In some embodiments, the reacting the compound of formula XX or a co-crystal, solvate, salt, or combination thereof is performed in a solvent (e.g., a second solvent) comprising toluene.

In some embodiments, the reacting the compound of formula XX or a co-crystal, solvate, salt, or combination thereof is carried out in a temperature range of from about −50° C. to about 50° C. In some embodiments, the reacting the compound of formula XX or a co-crystal, solvate, salt, or combination thereof is carried out in a temperature range of from about −5° C. to about 15° C., such as about 5° C.

In some embodiments, the process further comprises preparing the compound of formula XX or a co-crystal, solvate, salt, or combination thereof by a process comprising:
reducing a compound of formula XIX:

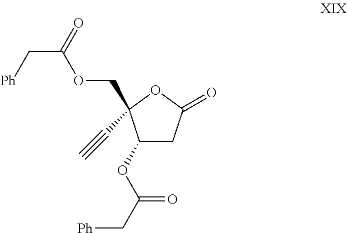 [SMILES: C#C[C@]1(COC(=O)Cc2ccccc2)OC(=O)C[C@@H]1OC(=O)Cc1ccccc1]

or a co-crystal, solvate, salt, or combination thereof, with
a reducing agent,
   to provide the compound of formula XX or a co-crystal, solvate, salt, or combination thereof.

In some embodiments, the reducing agent is selected from the group consisting of diisobutylaluminum hydride, sodium bis(2-methoxyethoxy)aluminum hydride, lithium aluminum hydride, lithium borohydride, and lithium tri-t-butoxyaluminum hydride. In some embodiments, the reducing agent is diisobutylaluminum hydride.

In some embodiments, the reducing is performed in a solvent (e.g., a third solvent) comprising an ether (e.g., tetrahydrofuran, 2-methyltetrahydrofuran, tert-butyl methyl ether, 1,4-dioxane, 1,2-dimethoxyethane, diglyme, dimethyl isosorbide), a hydrocarbon (e.g., toluene, xylene), or a combination thereof. In some embodiments, the reducing is performed in a solvent (e.g., a third solvent) comprising 1,2-dimethoxyethane.

In some embodiments, the reducing is carried out in a temperature range of from about −80° C. to about −30° C. In some embodiments, the reducing is carried out in a temperature range of from about −78° C. to about −30° C., or about −60° C. to about −40° C., such as about −50° C.

In some embodiments, the process further comprises preparing the compound of formula XIX or a co-crystal, solvate, salt, or combination thereof by a process comprising:
esterifying a compound of formula X:

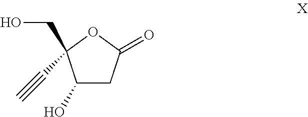

or a co-crystal, solvate, salt, or combination thereof, with
a second base, and
a second catalyst,
   to provide the compound of formula XIX or a co-crystal, solvate, salt, or combination thereof.

In some embodiments, the fourth base is an organic base or an inorganic base.

In some embodiments, the organic base is selected from the group consisting of N-methylimidazole, triethylamine, Hunig's base, pyridine, imidazole, DABCO, and DBU. In some embodiments, the fourth base is N-methylimidazole.

In some embodiments, the inorganic base comprises an alkali metal carbonate, alkaline earth metal carbonate, or alkali metal hydroxide. In some embodiments, the inorganic base is selected from the group consisting of Na₂CO₃, Cs₂CO₃, K₂CO₃, NaOH, and KOH. In some embodiments, the second catalyst comprises an aromatic base. In some embodiments, the second catalyst is selected from the group consisting of 4-dimethylaminopyridine, pyridine, imidazole, and 4-pyrrolidinylpyridine. In some embodiments, the third catalyst is 4-dimethylaminopyridine.

In some embodiments, the esterifying is performed in a solvent (e.g., a fourth solvent) comprising a polar aprotic solvent. For example, the polar aprotic solvent comprises N,N-dimethylformamide, N,N-dimethylacetamide, N-methyl-2-pyrrolidone, ethyl acetate, THF, 1,2-dimethoxyethane, acetonitrile, dichloromethane, or a combination thereof. In some embodiments, the esterifying is performed in a solvent (e.g., a fourth solvent) comprising 1,2-dimethoxyethane and acetonitrile.

In some embodiments, the esterifying is carried out in a temperature range of from about −20° C. to about 40° C. In some embodiments, the esterifying is carried out in a temperature range of from about −20° C. to about 0° C.

In some embodiments, the process further comprises preparing the compound of formula X or a co-crystal, solvate, salt, or combination thereof by a process comprising:
reacting a compound of formula IX:

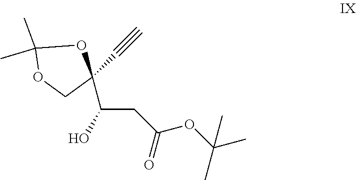 [SMILES: C#C[C@]1([C@@H](O)CC(=O)OC(C)(C)C)COC(C)(C)O1]

or a co-crystal, solvate, salt, or combination thereof, with
an acid,
to provide the compound of formula X or a co-crystal, solvate, salt, or combination thereof.

In some embodiments, the acid is a strong protic acid (e.g., HCl, sulfuric acid. hydrobromic acid, methanesulfonic acid, p-toluenesulfonic acid, trifluoroacetic acid) or a strong Lewis acid (e.g., boron tribromide, boron trichloride, boron trifluride, aluminum trichloride). In some embodiments, the acid is a strong protic acid. In some embodiments, the acid is HCl.

In some embodiments, the reacting the compound of formula IX or a co-crystal, solvate, salt, or combination thereof is performed in a solvent (e.g. a seventh solvent) comprising an ether (e.g., dioxane, diethyl ether, tetrahydrofuran, 2-methyltetrahydrofuran, 1,2-dimethoxyethane, tert-butyl methyl ether, diglyme, dimethyl isosorbide), halogenated solvents (e.g., 1,2-dichloroethane, chloroform, chlorobenzene, α,α,α-trifluorotoluene), polar aprotic solvent (e.g., N,N-dimethylformamide, N,N-dimethylacetamide, N-methyl-2-pyrrolidone), protic solvent (e.g., water, propionic acid, acetic acid), or a combination thereof. In some embodiments, the reacting the compound of formula IX or a co-crystal, solvate, salt, or combination thereof is performed in a solvent (e.g., a seventh solvent) comprising 1,2-dimethoxyethane.

In some embodiments, the reacting the compound of formula IX or a co-crystal, solvate, salt, or combination thereof is carried out in a temperature range of from about 30° C. to about 60° C. In some embodiments, the reacting the compound of formula IX or a co-crystal, solvate, salt, or combination thereof is carried out in a temperature range of from about 40° C. to about 50° C., such as about 45° C.

In some embodiments, the process further comprises preparing the compound of formula IX or a co-crystal, solvate, salt, or combination thereof by a process comprising desilylating a compound of formula VIII-A:

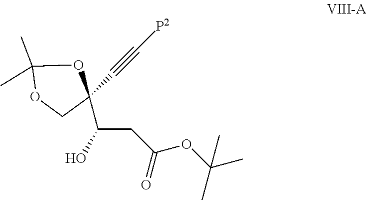

wherein P² is (R⁵)₃Si, wherein each R⁵ is independently C₁₋₆ alkyl;
or a co-crystal, solvate, salt, or combination thereof, with
a fluoride source,
to provide the compound of formula IX or a co-crystal, solvate, salt, or combination thereof.

In some embodiments, each R⁵ is independently C₁₋₃ alkyl. In some embodiments, P² is triisopropylsilyl or trimethylsilyl. In some embodiments, P² is triisopropylsilyl.

In some embodiments, the compound of formula VIII-A or a co-crystal, solvate, salt, or combination thereof is a compound of formula VIII:

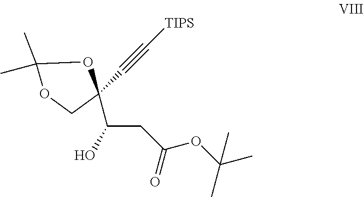

or a co-crystal, solvate, salt, or combination thereof.

In some embodiments, the fluoride source is selected from the group consisting of an alkali metal fluoride (e.g., NaF, CsF), an alkaline earth metal fluoride (e.g., CaF₂), hydrofluoric acid, pyridine-trihydrofluoride, tetrabutylammonium fluoride, and a hydrate thereof. In some embodiments, the fluoride source is tetrabutylammonium fluoride trihydrate.

In some embodiments, the desilylating is performed in a solvent (e.g., an eighth solvent) comprising an ether (e.g., dioxane, diethyl ether, tetrahydrofuran, 2-methyltetrahydrofuran, 1,2-dimethoxyethane, tert-butyl methyl ether, diglyme, dimethyl isosorbide), polar aprotic solvent (e.g., N,N-dimethylformamide, N,N-dimethylacetamide, N-methyl-2-pyrrolidone), or a combination thereof. In some embodiments, the desilylating is performed in a solvent (e.g., an eighth solvent) comprising tetrahydrofuran.

In some embodiments, the desilylating is carried out in a temperature range of from about −10° C. to about 35° C. In some embodiments, the desilylating is carried out in a temperature range of from about 5° C. to about 15° C. such as about 10° C.

In some embodiments, the process further comprises preparing the compound of formula VIII-A or a co-crystal, solvate, salt, or combination thereof by a process comprising: reacting a compound of formula VII-A:

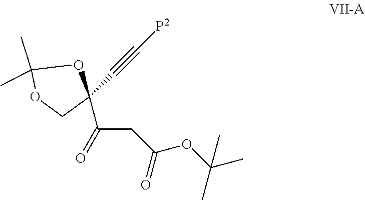

or a co-crystal, solvate, salt, or combination thereof, with
a hydrogen source, and
a fourth catalyst,
to provide the compound of formula VIII-A or a co-crystal, solvate, salt, or combination thereof.

In some embodiments, P² of formula VII-A is (R⁵)₃Si, wherein each R⁵ is independently C₁₋₆ alkyl. In some embodiments, each R⁵ is independently C₁₋₃ s alkyl. In some embodiments, P² of formula VII-A is triisopropylsilyl or trimethylsilyl. In some embodiments, P² of formula VII-A is triisopropylsilyl.

In some embodiments, the compound of formula VII-A or a co-crystal, solvate, salt, or combination thereof is a compound of formula VII:

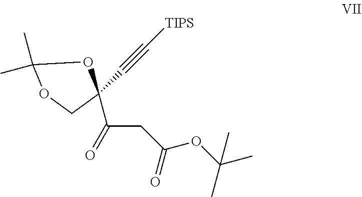

or a co-crystal, solvate, salt, or combination thereof.

In some embodiments, the hydrogen source is selected from the group consisting of formic acid, a salt of formic acid, sodium formate in water, a secondary alcohol (e.g., glycerol, 2-propanol), or Hantzsch ester. In some embodiments, the hydrogen source is a salt of formic acid. In some embodiments, the hydrogen source is a formic acid trimethylamine complex.

In some embodiments, the fourth catalyst is a transition metal catalyst. In some embodiments, the fourth catalyst comprises ruthenium, iron, osmium, cobalt, rhodium, iridium, nickel, palladium, gold, or a combination thereof. In some embodiments, the fourth catalyst is a transition metal catalyst having an N-heterocyclic carbine ligand. In some embodiments, the fourth catalyst is a ruthenium catalyst. In some embodiments, the fourth catalyst is N-[(1S,2S)-1,2-diphenyl-2-(2-(4-methylbenzyloxy)ethylamino)-ethyl]-4-methylbenzene sulfonamide (chloro) ruthenium (II), also known as (S,S)-Ts-DENEB®.

In some embodiments, the reacting the compound of formula VII-A or a co-crystal, solvate, salt, or combination thereof is performed in a solvent (e.g. a ninth solvent) comprising a polar aprotic solvent (e.g., N,N-dimethylformamide, N,N-dimethylacetamide, N-methyl-2-pyrrolidone), ether (e.g., tetrahydrofuran, 2-methyltetrahydrofuran, 1,2-dimethoxyethane, tert-butyl methyl ether, 1,4-dioxane, diglyme, dimethyl isosorbide), ketone (e.g., acetone, methyl ethyl ketone, methyl isobutyl ketone), halogenated solvent (e.g., 1,2-dichloroethane, chloroform, chlorobenzene, dichloromethane, α,α,α-trifluorotoluene), or a combination thereof. In some embodiments, the reacting the compound of formula VII-A or a co-crystal, solvate, salt, or combination thereof is performed in a solvent (e.g., a ninth solvent) comprising tetrahydrofuran.

In some embodiments, the reacting the compound of formula VII-A or a co-crystal, solvate, salt, or combination thereof is carried out in a temperature range of from about 30° C. to about 60° C. In some embodiments, the reacting the compound of formula VII-A or a co-crystal, solvate, salt, or combination thereof is carried out in a temperature range of from about 30° C. to about 40° C., such as about 35° C.

In some embodiments, the reacting the compound of formula VII-A or a co-crystal, solvate, salt, or combination thereof comprises:
preparing a first mixture comprising the compound of formula VII-A or a co-crystal, solvate, or combination thereof and the fourth catalyst;
preparing a second mixture comprising the hydrogen source; and
combining the first mixture and the second mixture to provide the compound of formula VIII-A or a co-crystal, solvate, salt, or combination thereof.

In some embodiments, the process further comprises preparing the compound of formula VII-A or a co-crystal, solvate, salt, or combination thereof by a process comprising:
reacting a compound of formula VI-A:

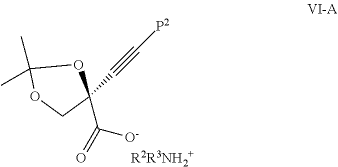

or a co-crystal, solvate, salt, or combination thereof,
wherein R² and R³ are independently selected from C₃-C₇ cycloalkyl, C₁₋₆ alkyl, and benzyl, with
a second acid,
a second activating reagent,
a malonic acid monoester, and
a fifth base,
to provide the compound of formula VII-A or a co-crystal, solvate, salt, or combination thereof.

In some embodiments, P² of formula VI-A is (R⁵)₃Si, wherein each R⁵ is independently C₁₋₆ alkyl. In some embodiments, each R⁵ is independently C₁₋₃ alkyl. In some embodiments, P² of formula VI-A is triisopropylsilyl or trimethylsilyl. In some embodiments, P² of formula VI-A is triisopropylsilyl.

In some embodiments, the compound of formula VI-A or a co-crystal, solvate, salt, or combination thereof is a compound of formula VI:

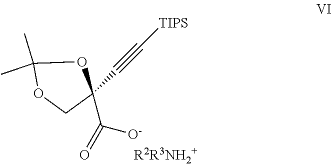

or a co-crystal, solvate, salt, or combination thereof.

In some embodiments, R² and R³ are independently C₃-C₇ cycloalkyl. In some embodiments, R² and R³ are cyclohexyl. In some embodiments, R² and R³ are independently C₁-C₆ alkyl. In some embodiments, R² and R³ are isopropyl. In some embodiments, R² and R³ are tert-butyl. In some embodiments, R² and R³ are benzyl. In some embodiments, R² and R³ are the same.

In some embodiments, the second acid is a protic acid. In some embodiments, the second acid is selected from the group consisting of citric acid, acetic acid, and hydrochloric acid. In some embodiments, the second acid is citric acid.

In some embodiments, the second activating agent is a carbonyl transfer reagent. In some embodiments, the second activating reagent is selected from the group consisting of 1,1′-carbonyldiimidazole, phosgene, triphosgene, and thionyl chloride. In some embodiments, the second activating agent is 1,1′-carbonyldiimidazole.

In some embodiments, the malonic acid monoester is selected from the group consisting of mono-tert-butyl malonate and mono-methyl malonate. In some embodiments, the malonic acid monoester is mono-tert-butyl malonate.

In some embodiments, the fifth base is a Grignard reagent. In some embodiments, the Grignard reagent comprises an alkyl magnesium halide or an aryl magnesium halide. For example, the fifth base comprises isopropyl magnesium chloride, isopropyl magnesium bromide, phenyl magnesium chloride, or phenyl magnesium bromide. In some embodiments, the fifth base is isopropyl magnesium chloride.

In some embodiments, the reacting the compound of formula VI-A or a co-crystal, solvate, salt, or combination thereof comprises:
preparing a first mixture comprising the second acid and the second activating reagent;
preparing a second mixture comprising the malonic acid monoester and the fifth base; and
combining the first mixture and the second mixture to provide the compound of formula VII-A or a co-crystal, solvate, salt, or combination thereof.

In some embodiments, the reacting the compound of formula VI-A or a co-crystal, solvate, salt, or combination thereof is performed in a solvent (e.g., a tenth solvent) comprising an ether (e.g., methyl-tert-butyl ether, tetrahydrofuran, 2-methyltetrahydrofuran, 1,2-dimethoxyethane, 1,4-dioxane, diglyme, dimethyl isosorbide). In some embodiments, the reacting the compound of formula VI-A or a co-crystal, solvate, salt, or combination thereof is performed in a solvent (e.g., a tenth solvent) comprising methyl-tert-butyl ether, tetrahydrofuran, 2-methyltetrahydrofuran, or a combination thereof.

In some embodiments, the reacting the compound of formula VI-A or a co-crystal, solvate, salt, or combination thereof is carried out in a temperature range of from about 0° C. to about 30° C. In some embodiments, the reacting the compound of formula VI-A or a co-crystal, solvate, salt, or combination thereof is carried out at a temperature of about 20° C.

In some embodiments, the process further comprises preparing the compound of formula VI-A or a co-crystal, solvate, salt, or combination thereof by a process comprising: oxidizing a compound of formula V-A:

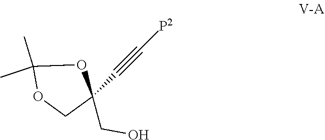

or a co-crystal, solvate, salt, or combination thereof, with
an oxidation reagent,
a fifth catalyst,
a sixth base, and
a salt forming reagent,
to provide the compound of formula VI-A or a co-crystal, solvate, salt, or combination thereof.

In some embodiments, P² of formula V-A is (R⁵)₃Si, wherein each R⁵ is independently C₁₋₆ alkyl. In some embodiments, each R⁵ is independently C₁₋₃ alkyl. In some embodiments, P² of formula V-A is triisopropylsilyl or trimethylsilyl. In some embodiments, P² of formula V-A is triisopropylsilyl.

In some embodiments, the compound of formula V-A or a co-crystal, solvate, salt, or combination thereof is a compound of formula V:

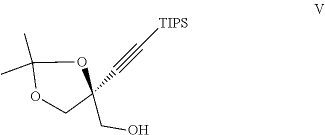

or a co-crystal, solvate, salt, or combination thereof.

In some embodiments, the oxidation reagent is selected from the group consisting of a hypervalent iodine reagent, activated DMSO (e.g., DMSO activated by oxalyl chloride, a carbodiimide, sulfur trioxide pyridine, acetic anhydride), a copper reagent (e.g., copper chloride, copper iodide, copper bromide), and a sulfur reagent (e.g., potassium peroxymonosulfate). In some embodiments, the oxidation reagent is a hypervalent iodine reagent. In some embodiments, the hypervalent iodine reagent is (diacetoxyiodo) benzene (DAIB), Dess-Martin periodinane, jodosylbenzene, or Koser's reagent. In some embodiments, the oxidation reagent is (diacetoxyiodo) benzene (DAIB).

In some embodiments, the fifth catalyst is selected from the group consisting of a nitrogen oxide, organic peroxide (e.g., benzoyl peroxide, di-tertbutyl peroxide), and azo radical initiator (e.g., azobisisobutylnitrile, 1,1′-azobis-(cyclohexanecarbonitrile). In some embodiments, the fifth catalyst is a nitrogen oxide. In some embodiments, the nitrogen oxide is selected from the group consisting of (2,2,6,6-tetramethylpiperidin-1-yl)oxidanyl (TEMPO), 4-hydroxy-2,2,6,6-tetramethylpoperidin-1-oxyl, and trimethylamine N-oxide. In some embodiments, the fifth catalyst is (2,2,6,6-tetramethylpiperidin-1-yl)oxidanyl (TEMPO).

In some embodiments, the sixth base comprises a phosphate or a carbonate. In some embodiments, the sixth base comprises an alkali metal phosphate (e.g., monosodium phosphate, trisodium phosphate), an alkali metal hydrogen phosphate (e.g., disodium hydrogen phosphate), an alkali metal carbonate (e.g., sodium carbonate, potassium carbonate, cesium carbonate), or a combination thereof. In some embodiments, the sixth base is disodium hydrogen phosphate.

In some embodiments, the salt forming reagent is a secondary amine having formula R²R³NH, wherein R² and R³ are as defined herein. In some embodiments, the salt forming reagent is selected from the group consisting of dicyclohexylamine, diisopropylamine, dibenzylamine, and di-tert-butylamine. In some embodiments, the salt forming reagent is dicyclohexylamine.

In some embodiments, the oxidizing is performed in an solvent (e.g. an eleventh solvent) comprising a polar aprotic solvent (e.g., N,N-dimethylformamide, N,N-dimethylacetamide, N-methyl-2-pyrrolidone, acetonitrile), ether (e.g., tetrahydrofuran, 2-methyltetrahydrofuran, tert-butyl methyl ether, 1,2-diemethoxyethane, 1,4-dioxane, diglyme, dimethyl isosorbide), ketone (e.g., acetone, methyl ethyl ketone, methyl isobutyl ketone), halogenated solvent (e.g., 1,2-dichloroethane, chloroform, chlorobenzene, dichloromethane, α,α,α-trifluorotoluene), water, or a combination thereof. In some embodiments, the oxidizing is performed in a solvent (e.g., an eleventh solvent) comprising acetonitrile and water.

In some embodiments, the oxidizing is carried out in a temperature range of from about 0° C. to about 50° C. In some embodiments, the oxidizing is carried out in a temperature range of from about 10° C. to about 30° C., such as 20° C.

In some embodiments, the process further comprises preparing the compound of formula V-A or a co-crystal, solvate, salt, or combination thereof by a process comprising:
reacting a compound of formula IV-A:

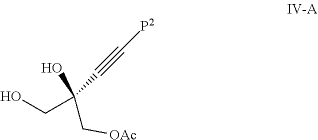

or a co-crystal, solvate, salt, or combination thereof, with
an acid catalyst,
a 1,2-diol protecting reagent, and
a seventh base,
to provide the compound of formula V-A or a co-crystal, solvate, salt, or combination thereof.

In some embodiments, P² of formula IV-A is (R⁵)₃Si, wherein each R⁵ is independently C₁₋₆ alkyl. In some embodiments, each R⁵ is independently C₁₋₃ alkyl. In some embodiments, P² of formula IV-A is triisopropylsilyl or trimethylsilyl. In some embodiments, P² of formula IV-A is triisopropylsilyl.

In some embodiments, the compound of formula IV-A or a co-crystal, solvate, salt, or combination thereof is a compound of formula IV:

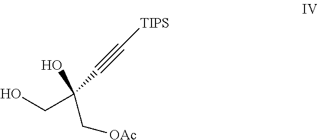

or a co-crystal, solvate, salt, or combination thereof.

In some embodiments, the acid catalyst is a weak acid having a pKa in a range of about 4 to about 6. In some embodiments, the acid catalyst is a weak acid having a pKa in the range of about 5. In some embodiments, the acid catalyst is selected from the group consisting of pyridinium para-toluenesulfonate (PPTS), acetic acid, zirconium (IV) chloride, and iodine. In some embodiments, the acid catalyst is pyridinium para-toluenesulfonate (PPTS).

In some embodiments, the 1,2-diol protecting reagent is selected from the group consisting of 2,2-dimethoxypropane, acetone, and isopropenyl acetate. In some embodiments, the 1,2-diol protecting reagent is 2,2-dimethoxypropane.

In some embodiments, the seventh base comprises an alkoxide or hydroxide. For example, the seventh base comprises an alkali metal alkoxide (e.g., sodium methoxide, sodium ethoxide), an alkali metal hydroxide (e.g., lithium hydroxide, sodium hydroxide, potassium hydroxide, cesium hydroxide), an alkali metal earth hydroxide (e.g., magnesium hydroxide, calcium hydroxide, barium hydroxide), or a combination thereof. In some embodiments, the seventh base comprises an alkali metal alkoxide. In some embodiments, the seventh base comprises sodium methoxide.

In some embodiments, the reacting the compound of formula IV-A or a co-crystal, solvate, salt, or combination thereof is performed in a solvent (e.g. a twelfth solvent) comprising an ether (e.g., methyl-tert-butyl ether, tetrahydrofuran. 2-methyltetrahydrofuran, cyclopentyl methyl ether, diisopropyl ether, di-n-butyl ether, 1,2-dimethoxyethane, diethylene glycol dimethyl ether, 1,4-dioxane, diglyme, dimethyl isosorbide), nitrile (e.g., acetonitrile, propionitrile, butyronitrile, benzonitrile), halogenated solvent (e.g., dichloromethane, 1,2-dichloroethane, chloroform, chlorobenzene, α,α,α-trifluorotoluene), or a combination thereof. In some embodiments, the reacting the compound of formula IV-A or a co-crystal, solvate, salt, or combination thereof is performed in a solvent (e.g., a twelfth solvent) comprising methyl-tert-butyl ether and acetonitrile.

In some embodiments, the reacting the compound of formula IV-A or a co-crystal, solvate, salt, or combination thereof is carried out in a temperature range of from about 20° C. to about 80° C. In some embodiments, the reacting the compound of formula IV-A or a co-crystal, solvate, salt, or combination thereof is carried out in a temperature range of from about 40° C. to about 60° C., such as about 50° C.

In some embodiments, the process further comprises preparing the compound of formula IV-A or a co-crystal, solvate, salt, or combination thereof by a process comprising:
reacting a compound of formula III-A:

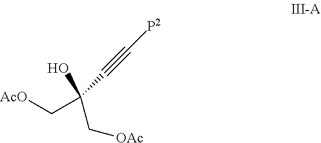

or a co-crystal, solvate, salt, or combination thereof, with
an eighth base, and
an enzyme,
to provide the compound of formula IV-A or a co-crystal, solvate, salt, or combination thereof.

In some embodiments, P² of formula III-A is (R⁵)₃Si, wherein each R⁵ is independently C₁₋₆ alkyl. In some embodiments, each R⁵ is independently C₁₋₃ alkyl. In some embodiments, P² of formula III-A is triisopropylsilyl or trimethylsilyl. In some embodiments, P² of formula III-A is triisopropylsilyl.

In some embodiments, the compound of formula III-A or a co-crystal, solvate, salt, or combination thereof is a compound of formula III:

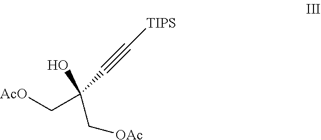

or a co-crystal, solvate, salt, or combination thereof.

In some embodiments, the eighth base comprises an alkali metal hydroxide and an alkali metal buffer. In some embodiments, the eighth base comprises an alkali metal hydroxide (e.g., NaOH, KOH) and an alkali metal phosphate monobasic aqueous buffer (e.g., KH₂PO₄, NaH₂PO₄). In some embodiments, the eighth base comprises potassium hydroxide and KH₂PO₄ aqueous buffer.

In some embodiments, the reacting the compound of formula III-A or a co-crystal, solvate, salt, or combination thereof is carried out at a pH in the range of from about 5 to about 7. In some embodiments, the reacting the compound of formula III-A or a co-crystal, solvate, salt, or combination thereof is carried out at a pH in the range of from about 5 to about 5.8.

In some embodiments, the enzyme is selected from the group consisting of Candida antartica lipase A (CALA) enzymes (e.g., NovoCorR ADL), Candida antarctica Lipase A immobilized on Immobead 150, recombinant enzymes derived from Aspergillus oryzae, Palatase® 20000 L, Novozym® 51032, and Lipozyme® TL 100 L, Lipozyme® CALB L. In some embodiments, the enzyme comprises Candida antarctica lipase A (CALA).

In some embodiments, the reacting the compound of formula III-A or a co-crystal, solvate, salt, or combination thereof is performed in a solvent (e.g., a thirteenth solvent) comprising an alcohol (e.g., methanol, ethanol, 1-propanol, 1-butanol, 2-butanol, tert-butanol), water, or a combination thereof. In some embodiments, the reacting the compound of formula III-A or a co-crystal, solvate, salt, or combination thereof is performed in a solvent (e.g., a thirteenth solvent) comprising methanol.

In some embodiments, the reacting the compound of formula III-A or a co-crystal, solvate, salt, or combination thereof is carried out in a temperature range of from about 5° C. to about 65° C. In some embodiments, the reacting the compound of formula III-A or a co-crystal, solvate, salt, or combination thereof is carried out in a temperature range of from about 20° C. to about 40° C., such as about 30° C.

In some embodiments, the process further comprises preparing the compound of formula III-A or a co-crystal, solvate, salt, or combination thereof by a process comprising:
reacting a compound of formula II:

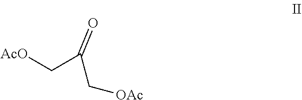 [SMILES: CC(=O)OCC(=O)COC(C)=O]

or a co-crystal, solvate, salt, or combination thereof, with
a compound of formula P²-acetylene, and
a ninth base,
to provide the compound of formula III-A or a co-crystal, solvate, salt, or combination thereof.

In some embodiments, the compound of formula P²-acetylene is (trimethylsilyl)acetylene or (triisopropylsilyl)acetylene. In some embodiments, the compound of formula P²-acetylene is (triisopropylsilyl)acetylene.

In some embodiments, the ninth base comprises an alkyl lithium (e.g., methyllithium, sec-butyllithium, isopropyllithium, tert-butyllithium), an aryl lithium (e.g., phenyllithium), a Grignard reagent (e.g., alkyl magnesium halide, aryl magnesium halide), lithium diisopropylamide, lithium hevamethyldisilazide, sodium hexamethyldisilazide, potassium hexamethyldisilazide, lithium amide, sodium amide, a metal hydride (e.g., sodium hydride, potassium hydride), an alkoxide (e.g., lithium tert-butoxide, sodium tert-butoxide, potassium tert-butoxide), or a combination thereof. In some embodiments, the ninth base comprises an alkyl lithium. In some embodiments, the ninth base comprises n-butyllithium.

In some embodiments, the reacting the compound of formula II or a co-crystal, solvate, salt, or combination thereof is performed in a solvent (e.g. a fourteenth solvent) comprising an ether (e.g., methyl-tert-butyl ether, tetrahydrofuran. 2-methyltetrahydrofuran, cyclopentyl methyl ether, diisopropyl ether, di-n-butyl ether, 1,2-dimethoxyethane, diethylene glycol dimethyl ether, 1,4-dioxane, diglyme, dimethyl isosorbide), hydrocarbon (e.g., toluene, n-hexane, hexanes, n-heptane, heptanes), or a combination thereof. In some embodiments, the reacting the compound of formula II or a co-crystal, solvate, salt, or combination thereof is performed in a solvent (e.g., a fourteenth solvent) comprising tetrahydrofuran.

In some embodiments, the reacting the compound of formula II or a co-crystal, solvate, salt, or combination thereof is carried out in a temperature range of from about −100° C. to about −40° C.

In some embodiments, the reacting the compound of formula II or a co-crystal, solvate, salt, or combination thereof comprises:
reacting the ninth base and the compound of formula P²-acetylene to form a mixture; and
combining the mixture with the compound of formula II to provide the compound of formula III-A or a co-crystal, solvate, salt, or combination thereof.

In some embodiments, the reacting the ninth base and the compound of formula P²-acetylene is carried out in a temperature range of about −60° C. to about −40° C., such as about −50° C. In some embodiments, the combining the mixture with the compound of formula II is carried out in a temperature range of about −70° C. to about −50° C., such as about −60° C.

In some embodiments, a compound of formula III-A:

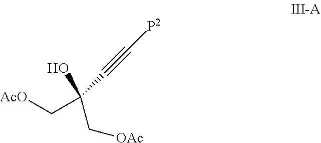

or a co-crystal, solvate, salt, or combination thereof is provided.

In some embodiments, a compound of formula IV-A:

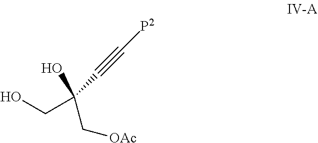

or a co-crystal, solvate, salt, or combination thereof is provided.

In some embodiments, a compound of formula V-A:

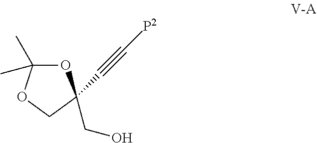

or a co-crystal, solvate, salt, or combination thereof is provided.

In some embodiments, a compound of formula VI-A:

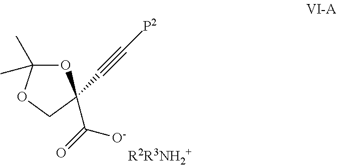

or a co-crystal, solvate, salt, or combination thereof is provided.

In some embodiments, a compound of formula VII-A:

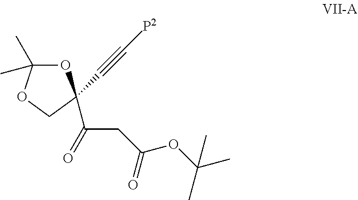

or a co-crystal, solvate, salt, or combination thereof is provided.

In some embodiments, a compound of formula VIII-A:

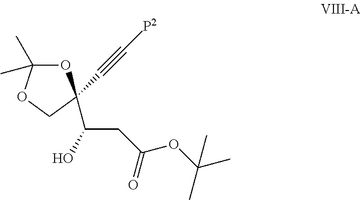

or a co-crystal, solvate, salt, or combination thereof is provided.

In some embodiments, a compound of formula IX:

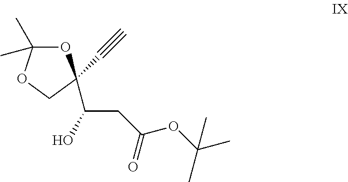 [SMILES: C#C[C@]1([C@@H](O)CC(=O)OC(C)(C)C)COC(C)(C)O1]

or a co-crystal, solvate, salt, or combination thereof is provided.

In some embodiments, a compound of formula X:

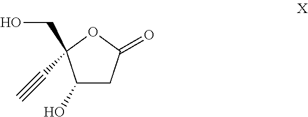

or a co-crystal, solvate, salt, or combination thereof is provided.

In some embodiments, a compound of formula XI:

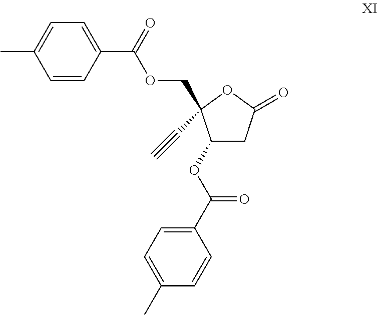 [SMILES: C#C[C@]1(COC(=O)c2ccc(C)cc2)OC(=O)C[C@@H]1OC(=O)c1ccc(C)cc1]

or a co-crystal, solvate, salt, or combination thereof is provided.

In some embodiments, a compound of formula XII:

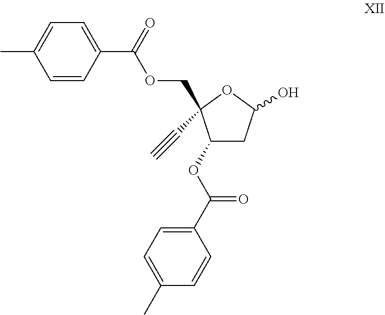 [SMILES: C#C[C@]1(COC(=O)c2ccc(C)cc2)OC(O)C[C@@H]1OC(=O)c1ccc(C)cc1]

or a co-crystal, solvate, salt, or combination thereof is provided.

In some embodiments, a compound of formula XIV:

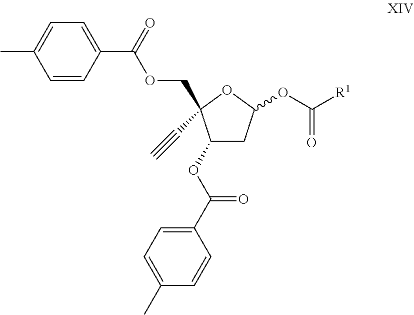

or a co-crystal, solvate, salt, or combination thereof is provided.

In some embodiments, a compound of formula XVI-A:

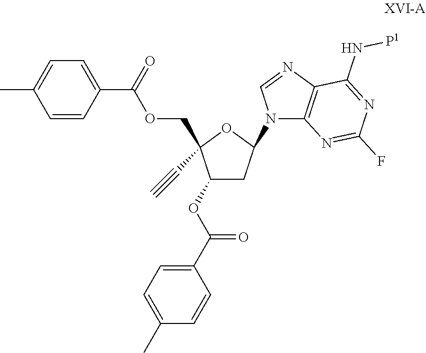

or a co-crystal, solvate, salt, or combination thereof is provided.

In some embodiments, a compound of formula XIX:

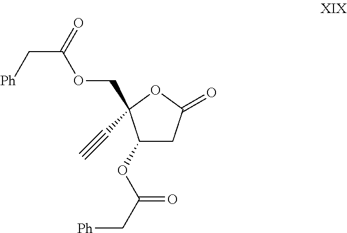 [SMILES: C#C[C@]1(COC(=O)Cc2ccccc2)OC(=O)C[C@@H]1OC(=O)Cc1ccccc1]

or a co-crystal, solvate, salt, or combination thereof is provided.

In some embodiments, a compound of formula XX:

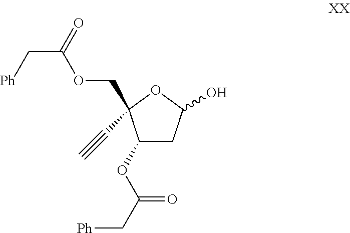 [SMILES: C#C[C@]1(COC(=O)Cc2ccccc2)OC(O)C[C@@H]1OC(=O)Cc1ccccc1]

or a co-crystal, solvate, salt, or combination thereof is provided.

In some embodiments, a compound of formula XXII:

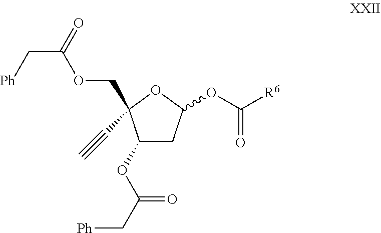

or a co-crystal, solvate, salt, or combination thereof is provided.

In some embodiments, provided herein is a process for preparing a compound of formula

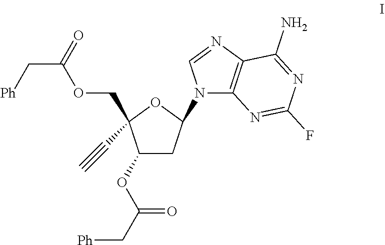 [SMILES: C#C[C@]1(COC(=O)Cc2ccccc2)O[C@@H](n2cnc3c(N)nc(F)nc32)C[C@@H]1OC(=O)Cc1ccccc1]

or a co-crystal, solvate, salt, or combination thereof, comprising:
   (a) esterifying a compound of formula X:

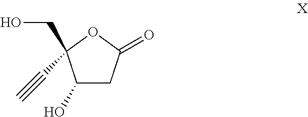

or a co-crystal, solvate, salt, or combination thereof, with
a fourth base,
a third catalyst, and
an acylating reagent,
   to provide a compound of formula XI:

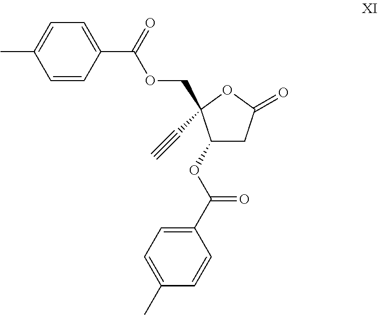 [SMILES: C#C[C@]1(COC(=O)c2ccc(C)cc2)OC(=O)C[C@@H]1OC(=O)c1ccc(C)cc1]

or a co-crystal, solvate, salt, or combination thereof.
(b) reducing the compound of formula XI or a co-crystal, solvate, salt, or combination thereof, with
a reducing agent,
   to provide a compound of formula XII:

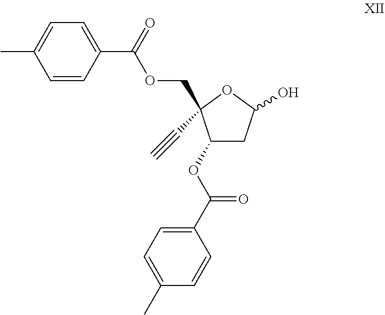 [SMILES: C#C[C@]1(COC(=O)c2ccc(C)cc2)OC(O)C[C@@H]1OC(=O)c1ccc(C)cc1]

or a co-crystal, solvate, salt, or combination thereof;
(c) reacting the compound of formula XII or a co-crystal, solvate, salt, or combination thereof, with
a compound selected from the group consisting of a compound of formula XIII-A:

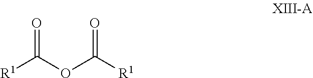

and a compound of formula XIII-B:

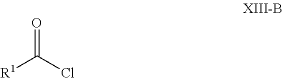

wherein R¹ is selected from C₁₋₄ alkyl, C₁₋₄ haloalkyl, C₁₋₄ alkoxy, benzyloxy, or C₆₋₁₀ aryl optionally substituted with 1 to 5 substituents independently selected from C₁₋₄ alkyl, C₁₋₄ haloalkyl, halo, C₁₋₄ alkoxy, or benzyloxy,
a third base, and
a second catalyst,
   to provide a compound of formula XIV:

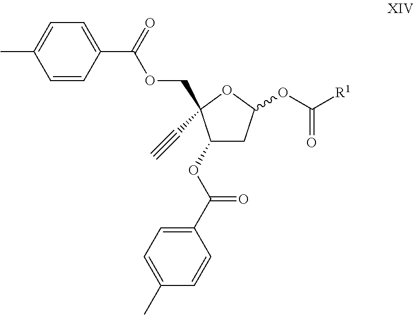

or a co-crystal, solvate, salt, or combination thereof;
(d) reacting the compound of formula XIV or a co-crystal, solvate, salt, or combination thereof, with a compound of formula XV:

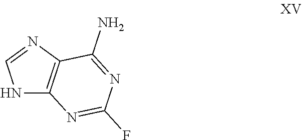 [SMILES: Nc1nc(F)nc2[nH]cnc12]

or a co-crystal, solvate, salt, or combination thereof,
a silylating reagent, and
a Lewis acid,
   to provide a compound of formula XVI-A:

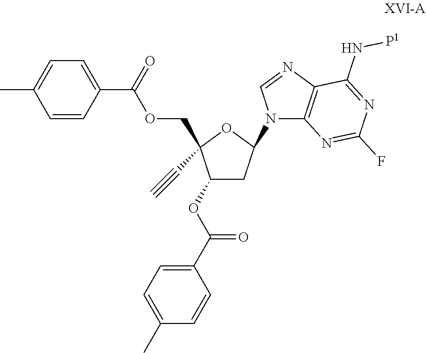

or a co-crystal, solvate, salt, or combination thereof,
wherein P¹ is H or (R⁴)₃Si, wherein each R⁴ is independently C₁₋₆ alkyl;
(e) reacting the compound of formula XVI or a co-crystal, solvate, salt, or combination thereof, with:
a de-acylation reagent, and
a second base,
   to provide a compound of formula XVII:

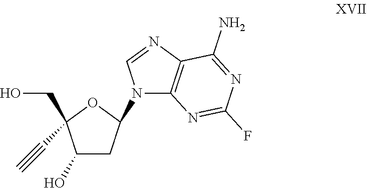 [SMILES: C#C[C@]1(CO)O[C@@H](n2cnc3c(N)nc(F)nc32)C[C@@H]1O]

or a co-crystal, solvate, salt, or combination thereof; and
(f) reacting the compound of formula XVII or a co-crystal, solvate, salt, or combination thereof, with
a compound of formula XVIII:

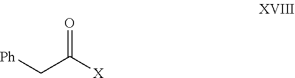

wherein X is selected from halo, anhydride, and OH, and
a base,
   to provide the compound of formula I or a co-crystal, solvate, salt, or combination thereof.

In some embodiments, provided herein is a process for preparing a compound of formula I:

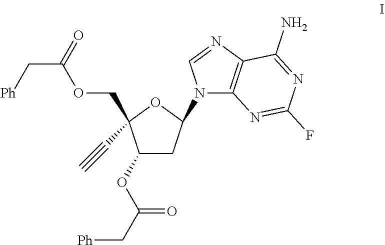 [SMILES: C#C[C@]1(COC(=O)Cc2ccccc2)O[C@@H](n2cnc3c(N)nc(F)nc32)C[C@@H]1OC(=O)Cc1ccccc1]

or a co-crystal, solvate, salt, or combination thereof, comprising:
(a) esterifying a compound of formula X:

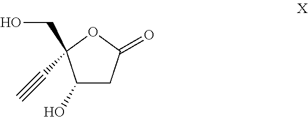

or a co-crystal, solvate, salt, or combination thereof, with
a second base, and
a second catalyst,
   to provide a compound of formula XIX:

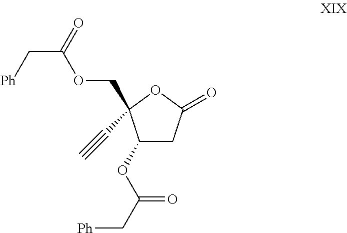 [SMILES: C#C[C@]1(COC(=O)Cc2ccccc2)OC(=O)C[C@@H]1OC(=O)Cc1ccccc1]

or a co-crystal, solvate, salt, or combination thereof;
(b) reducing the compound of formula XIX or a co-crystal, solvate, salt, or combination thereof, with
a reducing agent,
   to provide a compound of formula XX:

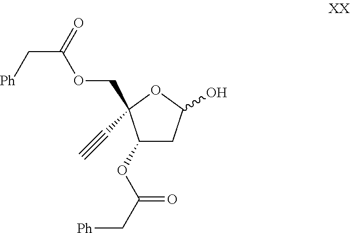 [SMILES: C#C[C@]1(COC(=O)Cc2ccccc2)OC(O)C[C@@H]1OC(=O)Cc1ccccc1]

or a co-crystal, solvate, salt, or combination thereof;
(c) reacting the compound of formula XX or a co-crystal, solvate, salt, or combination thereof, with
a compound selected from the group consisting of a compound of formula XXI-A:

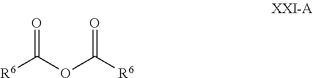

and a compound of formula XXI-B:

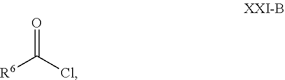

wherein R⁶ is selected from C₁₋₄ alkyl, C₁₋₄ haloalkyl, C₁₋₄ alkoxy, benzyloxy, or C₆₋₁₀ aryl optionally substituted with 1 to 5 substituents independently selected from C₁₋₄ alkyl, C₁₋₄ haloalkyl, halo, C₁₋₄ alkoxy, or benzyloxy,
a base, and
a catalyst,
   to provide a compound of formula XXII:

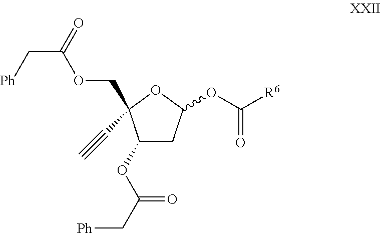

or a co-crystal, solvate, salt, or combination thereof; and
(d) reacting the compound of formula XXII or a co-crystal, solvate, salt, or combination thereof,
with a compound of formula XV:

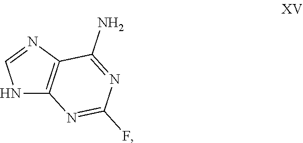 [SMILES: Nc1nc(F)nc2[nH]cnc12]

a silylating reagent, and
a Lewis acid,
   to provide the compound of formula I or a co-crystal, solvate, salt, or combination thereof.

The processes disclosed herein include, for example, alternative routes for the synthesis of compound I, or a co-crystal, solvate, salt, or combination thereof. The compound of formula X or a co-crystal, solvate, salt, or combination thereof can be used, for example, as an intermediate in any of the synthetic routes provided herein.

In some embodiments, the compound of formula X or a co-crystal, solvate, salt, or combination thereof is prepared by a process comprising:

(a) reacting a compound of formula II:

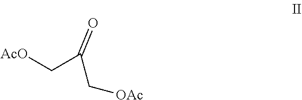 [SMILES: CC(=O)OCC(=O)COC(C)=O]

or a co-crystal, solvate, salt, or combination thereof, with
a compound of formula P²-acetylene, wherein P² is (R⁴)₃Si, wherein each R⁴ is independently C₁₋₆ alkyl, and
a ninth base,
   to provide a compound of formula III-A:

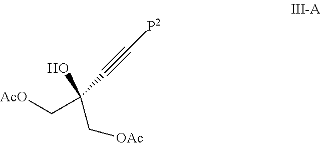

or a co-crystal, solvate, salt, or combination thereof;
(b) reacting the compound of formula III-A or a co-crystal, solvate, salt, or combination thereof, with
an eighth base, and
an enzyme,
   to provide a compound of formula IV-A:

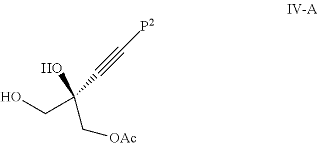

or a co-crystal, solvate, salt, or combination thereof;
(c) reacting the compound of formula IV-A or a co-crystal, solvate, salt, or combination thereof, with
an acid catalyst,
a 1,2-diol protecting reagent, and
a seventh base,
   to provide a compound of formula V-A:

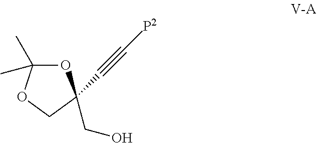

or a co-crystal, solvate, salt, or combination thereof;
(d) oxidizing the compound of formula V-A or a co-crystal, solvate, salt, or combination thereof, with
an oxidation reagent,
a fifth catalyst,
a sixth base, and
a salt forming reagent,
   to provide a compound of formula VI-A:

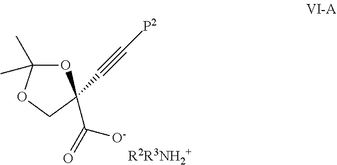

or a co-crystal, solvate, salt, or combination thereof,
wherein R² and R³ are independently selected from C₃-C₇ cycloalkyl, C₁₋₆ alkyl, and benzyl;
(e) reacting the compound of formula VI-A or a co-crystal, solvate, salt, or combination thereof, with
a second acid,
a second activating reagent,
a malonic acid monoester, and
a fifth base,
   to provide a compound of formula VII-A:

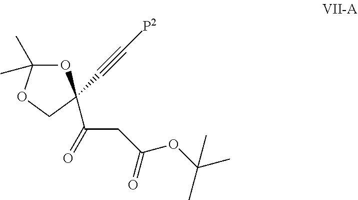

or a co-crystal, solvate, salt, or combination thereof;
(f) reacting the compound of formula VII-A or a co-crystal, solvate, salt, or combination thereof, with
a hydrogen source, and
a fourth catalyst,
   to provide a compound of formula VIII-A:

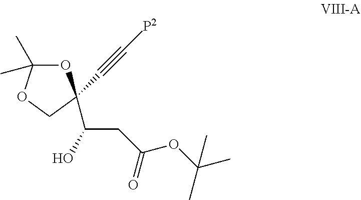

or a co-crystal, solvate, salt, or combination thereof;
(g) desilylating the compound of formula VIII-A or a co-crystal, solvate, salt, or combination thereof, with
a fluoride source,
   to provide a compound of formula IX:

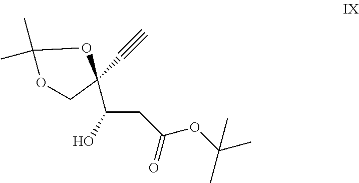 [SMILES: C#C[C@]1([C@@H](O)CC(=O)OC(C)(C)C)COC(C)(C)O1]

or a co-crystal, solvate, salt, or combination thereof; and
(h) reacting the compound of formula IX or a co-crystal, solvate, salt, or combination thereof, with
an acid,
   to provide the compound of formula X or a co-crystal, solvate, salt, or combination thereof.

In some embodiments, the current disclosure relates to the use of the compound of formula I for treating a Retroviridae viral infection including an infection caused by the HIV virus comprising administering a therapeutically effective amount of the compound of formula I to a subject in need thereof.

### EXAMPLES

Absolute stereochemistry was determined for compounds XI and XVII. All other stereochemistry shown throughout the examples is assigned based on extrapolation and literature precedence.

### Example 1. Preparation of (4S,5R)-5-ethynyl-4-hydroxy-5-(hydroxymethyl)dihydrofuran-2(3H)-one (X)

### Step 1. Preparation of 2-hydroxy-2-((triisopropylsilyl)ethynyl)propane-1,3-diyl diacetate (III)

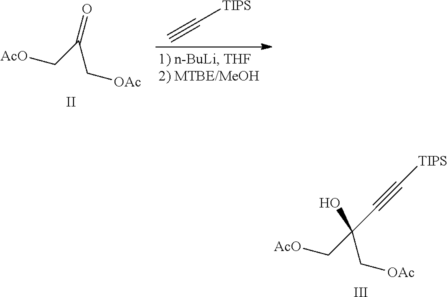

Diacetoxyacetone (II, 1.0 equiv., scaling factor) was charged to a vessel containing tetrahydrofuran (8 volumes) and cooled to about −60° C. TIPS-alkyne (TIPS-acetylene, 1.03 equiv.) and tetrahydrofuran (6 volumes) were charged to another vessel and cooled to about −60° C., and then n-butyllithium (1.0 equiv., 2.5 M in hexanes) was added over about 2 hours maintaining temperature below −40° C. The resulting reaction mixture was transferred to the reactor containing II solution over about 2 hours maintaining temperature below −50° C. The reaction was aged at about −60° C. for about 30 minutes and then quenched by slow addition of acetic acid (1.2 equiv.) maintaining temperature below −40° C. To the quenched reaction mixture was charged tert-butyl methyl ether (4 volumes) and the mixture was warmed to about 20° C. The reaction mixture was washed successively with 5 wt. % potassium bicarbonate in water (5 volumes) and 10 wt. % sodium chloride in water (5 volumes). The organic layer was concentrated under vacuum to about 4 volumes, then methanol (15 volumes) was added, and the mixture was concentrated under vacuum to about 4 volumes. The concentrate was diluted with methanol (5 volumes) and filtered to obtain a solution of III in methanol for use in the next step (Step 2).

### Step 2. Preparation of (R)-2-hydroxy-2-(hydroxymethyl)-4-(triisopropylsilyl)but-3-yn-1-yl acetate (IV)

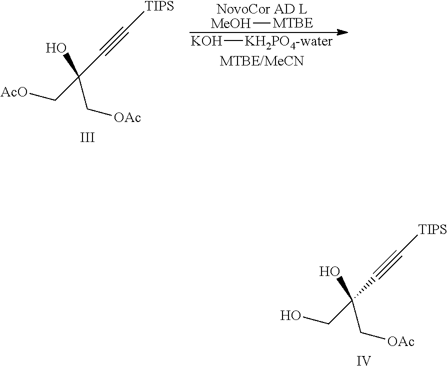

Phosphate buffer (0.1 M, pH=7.5, 1.8 volumes) including potassium phosphate monobasic and potassium hydroxide in water was charged to a reactor, warmed to about 30° C., and then NovoCorR AD L enzyme (1.2 g/g) was charged. The pH of the mixture was adjusted to about 5.8 with phosphoric acid, and then III solution in methanol (1.0 equiv., scaling factor) was charged. The pH of the mixture was adjusted to about 5.4 and the mixture was agitated at about 30° C. for about 15 hours. Tert-butyl methyl ether (4 volumes) was added, and the mixture was stirred for about 30 minutes. An aqueous solution of 15 wt. % sodium chloride (4 volumes) was charged, stirred for about 30 minutes, and then allowed to settle for at least 30 minutes. The bottom aqueous layer was discarded. The organic layer was filtered to remove emulsion and rinsed forward with tert-butyl methyl ether (1 volume). The filtrate was washed with a 15 wt % aqueous solution of sodium chloride (4 volumes) to afford the organic layer as a solution of IV in tert-butyl methyl ether.

### Step 3. Preparation of(S)-(2,2-dimethyl-4-((triisopropylsilyl)ethynyl)-1,3-dioxolan-4-yl)methanol (V)

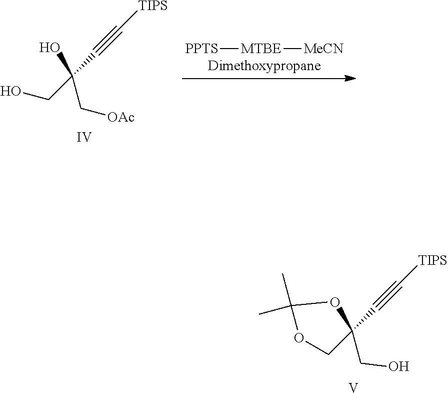

A solution of IV in tert-butyl methyl ether (1.0 equiv. scaling factor) was charged to a reactor, concentrated under vacuum to about 4 volumes and diluted with acetonitrile (10 volumes). The resulting mixture is concentrated to about 4 volumes and adjusted to about 20° C. 2,2-Dimethoxypropane (4 equiv.), tert-butyl methyl ether (5 volumes) and pyridinium p-toluenesulfonate (PPTS, 0.1 equiv.) were charged to the reactor, and, the contents were stirred at about 50° C. for about 2 hours, and then cooled to about 20° C. Sodium methoxide in methanol (25 wt. %, 0.4 equiv.) was charged and the contents were agitated at about 20° C. for about 15 minutes. The reaction mixture was washed successively with a 10 wt. % aqueous citric acid solution (5 volumes), a 15 wt. % aqueous sodium chloride solution (5 volumes) and water (2.5 volumes). The organic layer was concentrated under vacuum to about 4 volumes, diluted with acetonitrile (10 volumes), and then concentrated under vacuum to about 4 volumes to afford a solution of V in acetonitrile.

### Step 4. Preparation of dicyclohexylammonium(R)-2,2-dimethyl-4-((triisopropylsilyl)ethynyl)-1,3-dioxolane-4-carboxylate (VI-1)

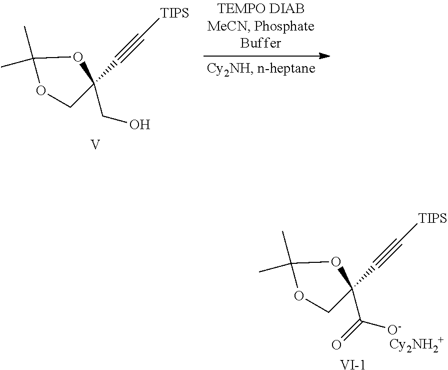

A solution of V (1 equiv., scaling factor) in acetonitrile was added to a reactor containing a solution of disodium hydrogen phosphate (3.1 equiv.) in water (4.0 volumes) and diacetoxyiodo benzene (DAIB, 2.4 equiv.). The reactor was then charged with (2,2,6,6-Tetramethylpiperidin-1-yl)oxidanyl (TEMPO, 0.2 equiv.) in portions, at a rate such as to keep the internal reactor temperature below 40° C. The internal reactor temperature was then adjusted to around 20° C., and the contents of the reactor were allowed to agitate for about 2 hours. Afterward, the reactor was charged with an aqueous solution of sodium sulfite (1.5 equiv. sodium sulfite in 3.4 volumes of water) to quench the reaction, and the quenched reactor contents were allowed to settle after stirring for about 30 minutes. The bottom aqueous layer was discarded, and dicyclohexylamine (1.55 equiv.) was charged to the reactor over a period of about 1 hour, and the reactor contents were stirred for about 2 hours. The resulting slurry was filtered, and the solids were washed with water (5.0 to 10 volumes) and heptane (4.0 volumes). The filter cake was then dried under vacuum at a jacket temperature of about 60° C. for approximately 18 hours to obtain VI-1.

¹H NMR (400 MHz, Chloroform-d) δ 4.33 (d, J=8.0 Hz, 1H), 4.24 (d, J=8.0 Hz, 1H), 2.97 (tt, J=11.7, 3.8 Hz, 2H), 2.09-1.94 (m, 4H), 1.77 (d, J=9.9 Hz, 5H), 1.66-1.47 (m, 9H), 1.42 (s, 3H), 1.28-1.13 (m, 6H), 1.06 (d, J=3.8 Hz, 21H).

This process was found to be reproducible and amenable for scale-up. The dicyclohexylamine salt VI-1 was identified as an isolable, stable compound. In addition, the process disclosed herein afforded the compound VI-1 with good purity and stereochemical control.

### Step 5. Preparation of tert-butyl(R)-3-(2,2-dimethyl-4-((triisopropylsilyl)ethynyl)-1,3-dioxolan-4-yl)-3-oxopropanoate (VII)

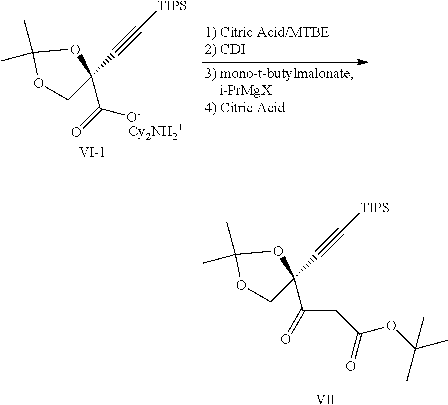

VI-1 (1.0 equiv., scaling factor), tert-butyl methyl ether (5 volumes) and 15 wt. % aqueous citric acid solution (5 volumes) were charged to a reactor and the contents were agitated for about 15 min then allowed to settle for about 15 min. The bottom aqueous layer was discarded. The organic layer was washed with a 15 wt. % aqueous citric acid solution (5 volumes) and then with a 10 wt. % aqueous sodium chloride solution (5 volumes). The organic layer was concentrated under vacuum at about 40° C. to about 2 volumes, and then diluted with 2-methyltetrahydrofuran (5 volumes). The reactor contents were concentrated under vacuum at about 40° C. to about 2 volumes, mixed with 2-methyltetrahydrofuran (2.5 volumes), and cooled at about 20° C. N,N-carbonyldiimidazole (1.2 equiv.) was charged to the reactor and the reaction mixture was agitated at about 20° C. for about 3 hours.

2-Methyltetrahydrofuran (2.5 volumes) and mono-tert-butyl malonate (1.4 equiv.) was charged to another reactor, and the resulting solution was cooled to about 0° C. A 2.0 M solution of isopropyl magnesium chloride in THF (2.8 equiv.) was charged to the reactor maintaining the reactor contents at about 15° C. The temperature was then adjusted to about 20° C. and the reaction mixture agitated for about 3 hours.

The contents in the first reactor were cooled to about 0° C. and the contents of the second reactor were transferred to first reactor maintaining temperature at about 15° C. The resulting reaction mixture was adjusted to about 20° C. and aged for about 12 hours. The reaction was then quenched by addition of 10 wt. % aqueous citric acid solution (10 volumes) and the mixture was agitated at about 20° C. for about 30 minutes and then allowed to settle for phase separation. The bottom aqueous layer was discarded. The organic layer was washed with a 5 wt. % aqueous NaHCO₃ solution (5 volumes), and then with a 10 wt. % aqueous NaCl solution (10 volumes). The organic layer was concentrated under vacuum at about 40° C. to about 2 volumes, diluted with tetrahydrofuran (10 volumes) and concentrated again under vacuum to about to afford a solution of VII in tetrahydrofuran.

The decarboxylative Claisen procedure as described herein was found to provide good scalability, yield, and product quality.

### Step 6. Preparation of tert-butyl(S)-3-((R)-2,2-dimethyl-4-((triisopropylsilyl)ethynyl)-1,3-dioxolan-4-yl)-3-hydroxypropanoate (VIII)

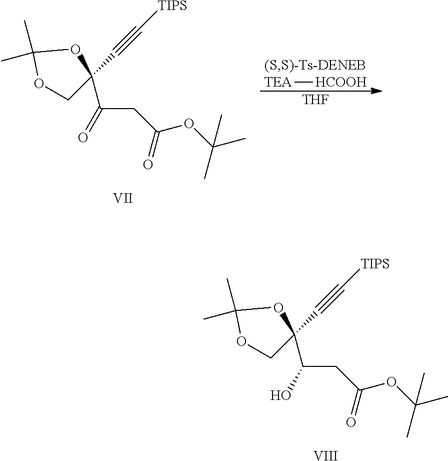

VII (1.0 equiv., scaling factor), tetrahydrofuran (9.0 volumes) and then (S,S)-Ts-DENEB (0.005 equiv.) were charged to a reactor. The reactor contents were agitated and was cooled to about 15° C. Another reactor was charged with tetrahydrofuran (1.0 volume) and triethylamine (1.0 equiv.), the mixture was cooled to about 0° C., and then formic acid (2.5 equiv.) was charged maintaining the reactor contents at about 0° C. Thereafter, the contents of the second reactor were transferred to the first reactor containing VII maintaining the reaction mixture at about 15° C. The resulting mixture was heated to about 35° C. and agitated for about 18 hours. The reaction mixture was then cooled to about 10° C. and the reactor was charged with tert-butyl methyl ether (10) volumes) and 10 wt. % aqueous citric acid solution (7.0 volumes). The biphasic mixture was agitated for about 30 minutes and then allowed to settle for phase separation. The lower aqueous phase was discarded. The organic phase was washed with a 10 wt % aqueous potassium bicarbonate solution (5.0 volumes) and then with a 10 wt. % aqueous sodium chloride solution (7.0 volumes). Activated carbon (Darco, 0.25 wt/wt) was charged and the resulting mixture was agitated at about 10° C. for about 1 hour. Then the reactor content was filtered, and the filter cake was washed with tert-butyl methyl ether (2.0 volumes), the combined organic phase was concentrated under vacuum to afford VIII.

### Step 7. Preparation of tert-butyl(S)-3-((R)-4-ethynyl-2,2-dimethyl-1,3-dioxolan-4-yl)-3-hydroxypropanoate (IX)

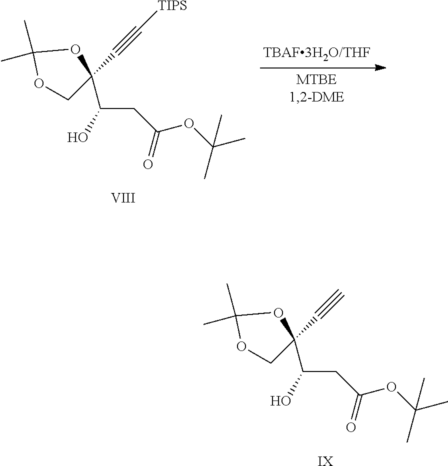

To VIII (1.0 equiv., scaling factor) in a reactor was charged tetrahydrofuran (5.0 volumes), and the contents were cooled to about 10° C. A 1.0 M tetrabutylammonium fluoride (TBAF) solution in tetrahydrofuran (1.1 equiv.) was charged and the reaction mixture was aged at about 10° C. for about 1 hour. The finding that this reaction is not sensitive to water content allowed for the preparation of the TBAF solution by dissolving readily available TBAF trihydrate in tetrahydrofuran. The reaction mixture was diluted with tert-butyl methyl ether (10 volumes) and washed twice with water (5.0 volumes each). The organic phase was filtered and concentrated under reduced pressure to afford IX.

### Step 8. (4S,5R)-5-ethynyl-4-hydroxy-5-(hydroxymethyl)dihydrofuran-2(3H)-one (X)

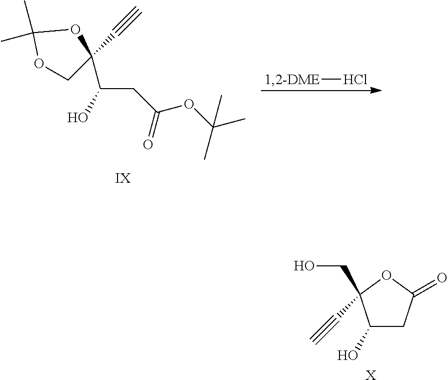

To IX (1.0 equiv., scaling factor) in a reactor was charged 1,2-dimethoxyethane (6.0 volumes). The reactor was then charged with concentrated hydrochloric acid (5.0 volumes) over a period of about 30 minutes, the reactor content was heated to about 45° C. and aged for about 2 hours. The reaction mixture was cooled to about 20° C. and concentrated to about 2 volumes. Acetonitrile (10 volumes) was added, then the mixture was concentrated to about 2 volumes. Thereafter, another portion of acetonitrile (10 volumes) was charged, and the mixture was concentrated to about 1.5 volumes to afford X.

¹H NMR (400 MHz, DMSO-d₆): 5.86 (s, 1H), 5.59 (s, 1H), 4.41-4.38 (m, 1H), 3.77 (s, 1H), 3.64-3.67 (m, 2H), 2.94 (dd, J=16 Hz, 8 Hz, 1H), 2.41 (dd, J=16 Hz, 4 Hz, 1H).

### Example 2. Preparation of (2R,3S,5R)-5-(6-amino-2-fluoro-9H-purin-9-yl)-2-ethynyl-2-((2-phenylacetoxy)methyl)tetrahydrofuran-3-yl 2-phenylacetate (I)

### Step 1. Preparation of (2R,3S)-2-ethynyl-2-(((4-methylbenzoyl)oxy)methyl)-5-oxotetrahydrofuran-3-yl 4-methylbenzoate (XI)

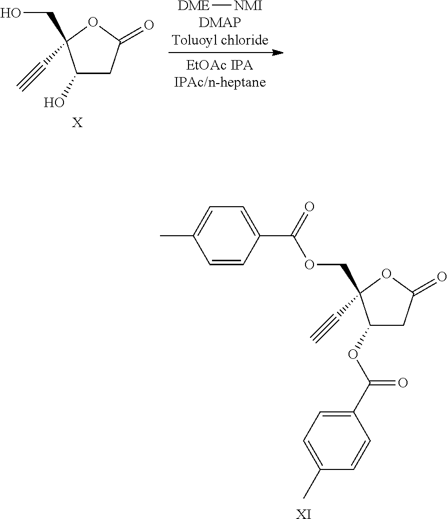

X (1.0 equiv., scaling factor) was mixed with 1,2-dimethoxyethane (8.0 volumes) and acetonitrile (1.0 volume) in a reactor and the resulting solution was cooled to about −20° C. The reactor was then charged with 4-dimethylaminopyridine (0.05 equiv.) followed by 1-methylimidazole (2.8 equiv.). Thereafter, 4-methylbenzoyl chloride (2.2 equiv.) was charged over a period of about 30 minutes, maintaining the reactor content below 0° C. The reaction mixture was then aged at about 0° C. for about 18 hours. The reactor was then charged with water (6.0 volumes) and ethyl acetate (10 volumes), the biphasic mixture was agitated for about 30 minutes at about 20° C., and then allowed to settle for phase separation. The bottom layer (aqueous phase) was discarded. The organic phase was concentrated under vacuum to about 3 volumes, then diluted with 2-propanol (10 volumes). The resulting mixture was agitated at about 40° C. for about 3 hours, then slowly cooled to about 10° C., over about 3 hours, and aged at about 10° C. for about 18 hours. The resulting slurry was filtered, the filter cake was washed with a 9:1 mixture heptane and isopropyl acetate (2 volumes) and dried under vacuum at about 40° C. for about 24 hours to obtain XI.

¹H NMR (400 MHz, DMSO-d6): δ 7.99-7.92 (m, 4H), 7.42-7.39 (m, 4H), 5.91 (dd, J=8 Hz, 4 Hz, 1H), 4.72 (m, 2H), 4.01 (s, 1H), 3.44 (dd, J=20 Hz, 8 Hz, 1H), 3.09 (dd, J=16 Hz, 4 Hz, 1H), 2.43 (s, 6H).

### Step 2. Preparation of (2R,3S)-2-ethynyl-5-hydroxy-2-(((4-methylbenzoyl)oxy)methyl)tetrahydrofuran-3-yl 4-methylbenzoate (XII)

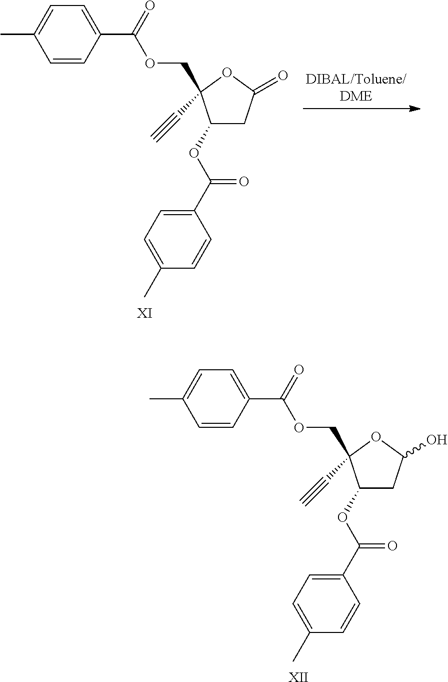

XI (1.0 equiv., scaling factor) and 1,2-dimethoxyethane (DME, 8.0 volumes) were charged to a reactor. The resulting solution was cooled to about −55° C. Diisobutylaluminum hydride (DIBAL) in toluene solution (1.90 equiv.) was charged slowly to the reactor maintaining the reactor contents below about −45° C. The resulting reaction mixture was diluted with pre-cooled (about −55° C.) toluene (8.0 volumes) and then quenched by slow addition of acetic acid (5 equiv.) maintaining reactor contents below about −20° C. The reaction mixture was washed with 1M H₂SO₄ aq. (7.0 volumes) and 10 wt. % NaCl aq. (7.0 volumes) and then concentrated under vacuum to about 3 volumes. Toluene (7.0 volumes) was charged. The resulting mixture was cooled to about 5° C. and agitated overnight. The resulting slurry was filtered and rinsed with toluene (1.5 volumes). The solution of XII in toluene was carried forward into next step (Step 3).

The process described herein was found to achieve the desired selective reduction to the lactol XII in a reproducible manner.

### Step 3. Preparation of (2R,3S)-5-acetoxy-2-ethynyl-2-(((4-methylbenzoyl)oxy)methyl)tetrahydrofuran-3-yl 4-methylbenzoate (XIV-1)

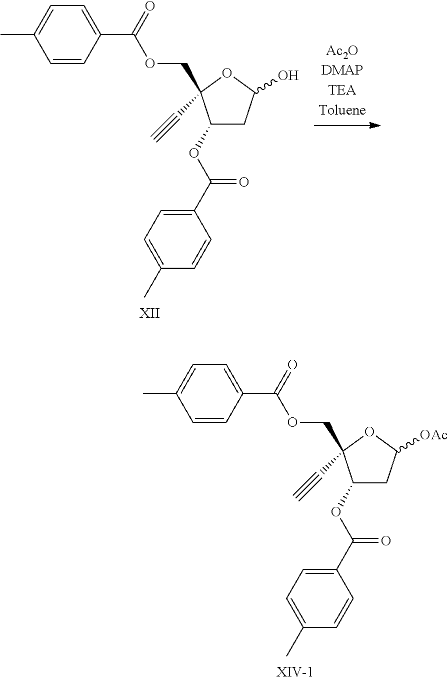

To a cooled solution of XII (about 0° C.) in toluene were charged 4-dimethylaminopyridine (DMAP, 0.05 equiv.) and triethylamine (1.25 equiv.) followed by slow addition of acetic anhydride (1.25 equiv.) maintaining reactor contents below about 5° C. The reaction mixture was then quenched with water (0.4 volumes) and washed successively with 10 wt. % citric acid aq. (5.0 volumes), 5 wt. % NaHCO; aq. (5.0 volumes) and water (5.0 volumes). The organic layer was concentrated under vacuum to about 2 volumes. Acetonitrile (7.0 volumes) was charged. The solution of XIV-1 in acetonitrile was carried forward into next step.

### Step 4. Preparation of (2R,3S,5R)-2-ethynyl-5-(2-fluoro-6-((trimethylsilyl) amino)-9H-purin-9-yl)-2-(((4-methylbenzoyl)oxy)methyl)tetrahydrofuran-3-yl 4-methylbenzoate (XVI)

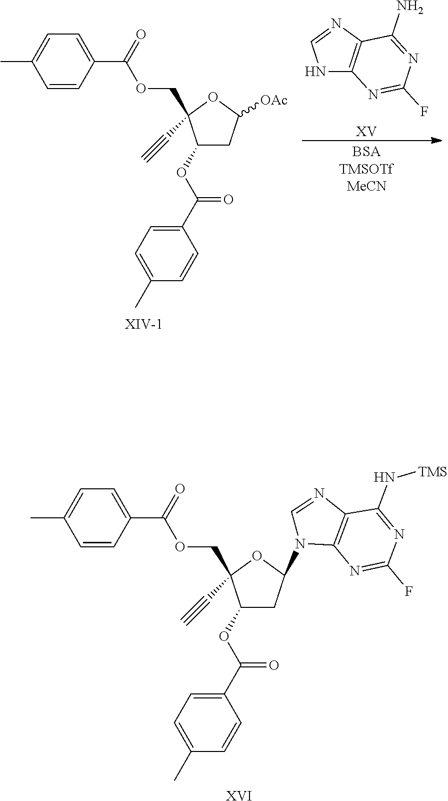

2-Fluoroadenine (XV, 1.1 equiv.), acetonitrile (10.0 volumes) and bis(trimethylsilyl)acetamide (BSA, 3.0 equiv.) were charged to a reactor. The reaction mixture was heated and maintained at about 72° C. for at least 1 hour. The reaction mixture was then cooled to about 20° C. and trimethylsilyl trifluoromethanesulfonate (TMSOTf, 1.1 equiv.) was charged. The reaction mixture was heated to about 72° C., and the solution of XIV-1 (1.0 equiv., scaling factor) in acetonitrile was added slowly to the reaction mixture maintaining the reactor contents above 70° C. over about 1 hour. Additional BSA (1.0 equiv.) was charged, and the reaction mixture was concentrated under vacuum to about 11 volumes maintaining reactor contents above 60° C. The mixture was then seeded with XVI (0.0007 equiv.) seeds, agitated at about 60° C. for about 1 hour and then cooled slowly to about 5° C. and stirred. The resulting slurry was filtered, the filter cake was washed with MTBE (2×1.5 volumes) and dried under vacuum at 50° C. to obtain XVI.

¹H NMR (400 MHz, DMSO-d₆): δ 8.36 (s, 1H), 7.90 (br, 2H), 7.97 (m, 2H), 7.83 (m, 2H), 7.38 (m, 2H), 7.28 (m, 2H), 6.53 (dd, 6.7 Hz, 1H), 6.09 (dd, 5.3 and 6.9 Hz, 1H), 4.70 (d, 11.5 Hz, 1H), 4.56 (d, 11.5 Hz, 1H), 3.80 (s, 1H), 3.35 (ddd, 14.1 Hz, 7.1 Hz, 6.9 Hz, 1H), 2.84 (ddd, 14.1 Hz, 7.0 Hz, 5.5 Hz, 1H), 2.41 (s, 3H), 2.37 (s, 3H), ¹³C NMR (126 MHz, DMSO-d₆): δ 165.0, 164.7, 158.6, 157.7, 150.2, 144.2, 143.9, 140.2, 129.5, 129.4, 129.2, 126.4, 126.3, 117.7, 82.8, 81.2, 80.0, 78.4, 73.6, 65.7, 34.5, 21.21, 21.20

### Step 5. Preparation of 4′-ethynyl-2-fluoro-2′-deoxyadenosine (islatravir, XVII)

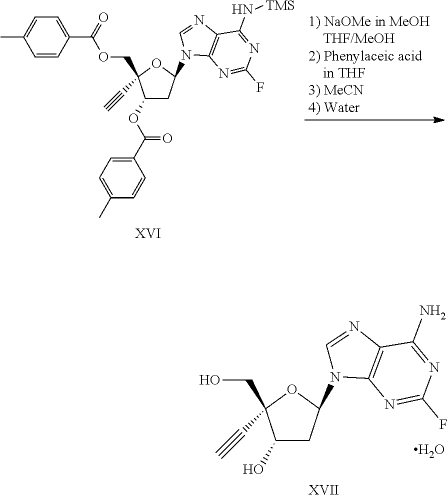

XVI (1 equiv., scaling factor) and THF (10.0 volumes) were charged to a reactor. The reactor contents were agitated to generate a clear solution, the resulting solution was cooled to about 0° C. A dilute solution of sodium methoxide in methanol was prepared by mixing pre-cooled (about 0° C.) methanol (5.0 volumes) with 25 wt. % sodium methoxide in methanol (0.03 equiv.) in another vessel. This solution was then charged to the XVI solution and the resulting reaction mixture was agitated at about 0° C. The reaction was quenched with a 1.0M phenylacetic acid solution in THE (containing 0.045 equiv. phenylacetic acid) at about 0° C., and then warmed to about 20° C. After agitating for about 1 hour, the reaction mixture was passed through a polish filter and the filtrate concentrated under vacuum to about 2 volumes. The concentrate was then diluted with about 5 volumes of 10 wt. % water in acetonitrile, and the resulting mixture was agitated for about 1 hour at about 40° C. The mixture was then concentrated under vacuum to about 2 volumes, and the resulting concentrate was diluted again with about 5 volumes of 10 wt. % water in acetonitrile. The resulting mixture was agitated for about 1 hour at about 40° C., and then, toluene (8.0 volumes) was charged to the mixture, and the reactor contents were cooled slowly to about 10° C. and stirred. The slurry was filtered, and the filter cake were washed with toluene (2×2.9 volumes), and then dried under vacuum at about 40° C. to obtain XVII.

¹H NMR (400 MHz, DMSO-d6): δ 8.34 (s, 1H), 7.88 (s, 2H), 6.29-6.26 (m, 1H), 5.62 (d, J=4.0 Hz, 1H), 5.35 (t, J=8 Hz, 1H), 4.62-4.60 (m, 1H), 3.71-3.67 (m, 1H), 3.62-3.54 (m, 1H), 3.52 (s, 1H), 2.76-2.72 (m, 1H), 2.50-2.43 (m, 1H).

### Step 6. (2R,3S,5R)-5-(6-amino-2-fluoro-9H-purin-9-yl)-2-ethynyl-2-(2-phenylacetoxy)methyl)tetrahydrofuran-3-yl 2-phenylacetate (I)

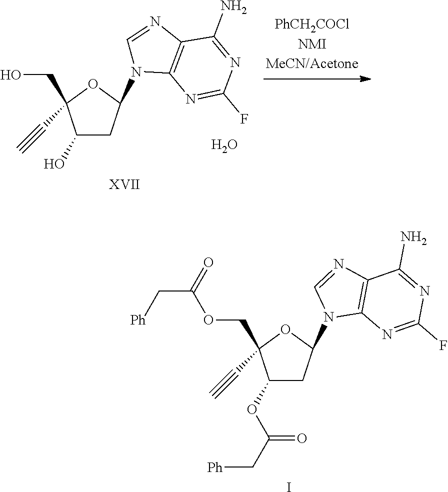

XVII (1 equiv., scaling factor) was charged to a reactor, followed by acetone (8.0 volumes) and acetonitrile (2.0 volumes). The reactor contents were agitated and N-methylimidazole (4.2 equiv.) was charged. The resulting solution was cooled to about −10° C., then treated with a solution of phenylacetyl chloride (3.8 equiv.) in acetonitrile (6.0 volumes) at about −5 to 0° C. After the addition of the phenylacetyl chloride solution, the reactor was warmed to about 10° C., then agitated. The reactor was charged with water (1.0 volume,), then was warmed to about 40° C. The reactor was then charged with water (5.0 volumes,), and the reactor contents were polish filtered into a reactor. The filtrate was cooled to about 30° C., then charged with I seeds (0.008 equiv.) and then the mixture was agitated for about 1 hour. Thereafter, the reactor was charged with water (10.0 volumes.) over about 1 hour. The reactor temperature was then cooled to about 10° C. The resulting slurry agitated and then filtered, rinsed with water (6.0 volumes) and 2-propanol (4.0 volumes). The solids were then dried under vacuum at about 50° C. to obtain I.

¹H NMR (400 MHz, Chloroform-d) δ 7.75 (d, J=1.4 Hz, 1H), 7.41-7.19 (m, 11H), 6.45 (s, 1H), 6.34 (t, J=6.7 Hz, 1H), 5.54 (dd, J=6.8, 4.6 Hz, 1H), 4.50 (d, J=12.0 Hz, 1H), 4.41 (d, J=12.0 Hz, 1H), 3.72 (s, 2H), 3.68 (s, 2H), 2.67-2.53 (m, 2H), 2.44 (d, J=0.8 Hz, 1H).

### Example 3. Alternate preparation of (2R,3S,5R)-5-(6-amino-2-fluoro-9H-purin-9-yl)-2-ethynyl-2-((2-phenylacetoxy)methyl)tetrahydrofuran-3-yl 2-phenylacetate (I)

### Step 1. Preparation of (2R,3S)-2-ethynyl-5-oxo-2-((2-phenylacetoxy)methyl)tetrahydrofuran-3-yl 2-phenylacetate (XIX)

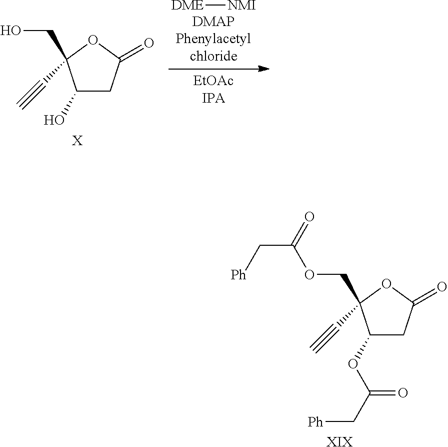

X (1.0 equiv., scaling factor) was mixed with 1,2-dimethoxyethane (8.0 volumes) and acetonitrile (1.0 volume) in a reactor and the resulting solution was cooled to about −20° C. The reactor was then charged with 4-dimethylaminopyridine (0.05 equiv.) followed by 1-methylimidazole (2.8 equiv.). Thereafter, phenylacetyl chloride (2.5 equiv.) was charged over a period of about 30 minutes maintaining the reactor content below 0° C. The reaction mixture was then aged at about 0° C. for about 18 hours, until the reaction was deemed complete. The reactor was then charged with water (6.0 volumes) and ethyl acetate (10 volumes), the biphasic mixture was agitated for about 30 minutes at about 20° C., and then allowed to settle for phase separation. The bottom layer (aqueous phase) was discarded. The organic phase was concentrated under vacuum, and the crude product was purified by column chromatography.

¹H NMR (400 MHz, DMSO-d6): δ 7.39-7.30 (m, 10H), 5.51 (dd, J=8 Hz, 4 Hz, 1H), 4.41 (m, 2H), 4.01 (s, 1H), 3.79 (m, 4H), 3.10 (dd, J=20 Hz, 8 Hz, 1H), 2.73 (dd, J=20 Hz, 4 Hz, 1H).

### Step 2. Preparation of (2R,3S)-2-ethynyl-5-hydroxy-2-((2-phenylacetoxy)methyl)tetrahydrofuran-3-yl 2-phenylacetate (XX)

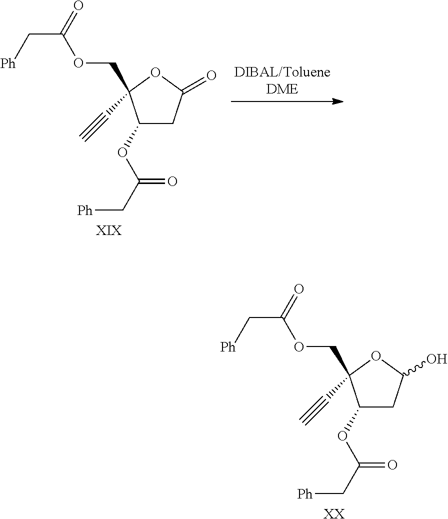

XIX (1.0 equiv., scaling factor) and 1,2-dimethoxyethane (DME, 8.0 volumes) were charged to a reactor. The resulting solution was cooled to about −55° C. Diisobutylaluminum hydride (DIBAL) in toluene solution (1.90 equiv.) was charged slowly to the reactor maintaining the reactor contents below about −45° C. The resulting reaction mixture was quenched with acetic acid (S equiv.) maintaining reactor contents below about −20° C. The reaction mixture was washed with IM H₂SO₄ aq. (7.0 volumes) and 10 wt. % NaCl aq. (7.0 volumes) and then concentrated under vacuum to about 3 volumes. Toluene (7.0 volumes) was charged. The solution of crude XX in toluene was carried forward into next step.

### Step 3. Preparation of (2R,3S)-5-acetoxy-2-ethynyl-2-((2-phenylacetoxy)methyl)tetrahydrofuran-3-yl 2-phenylacetate (XXII-1)

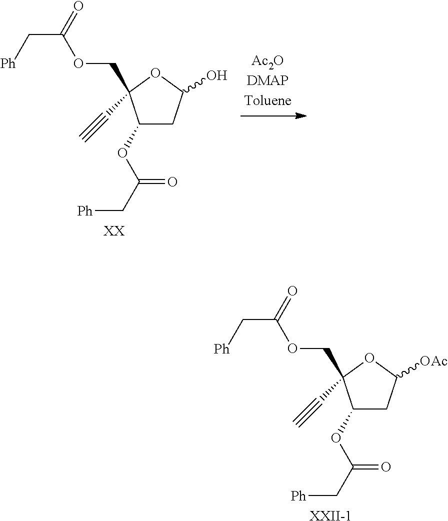

To a cooled solution of XX (about 0° C.) in toluene were charged 4-dimethylaminopyridine (DMAP, 0.05 equiv.) and triethylamine (1.25 equiv.) followed by slow addition of acetic anhydride (1.25 equiv.) maintaining reactor contents below about 5° C. The reaction mixture was then quenched with water (0.4 volumes) and washed successively with 10 wt. % citric acid aq. (5.0 volumes), 5 wt. % NaHCO₃ aq. (5.0 volumes) and water (5.0 volumes). The organic layer was concentrated under vacuum to about 2 volumes. Acetonitrile (7.0 volumes) was charged. The solution of crude XXII-1 in acetonitrile was carried forward into next step.

### Step 4. (2R,3S,5R) -5-(6-amino-2-fluoro-9H-purin-9-yl)-2-ethynyl-2-(2-phenylacetoxy)methyl)tetrahydrofuran-3-yl 2-phenylacetate (I)

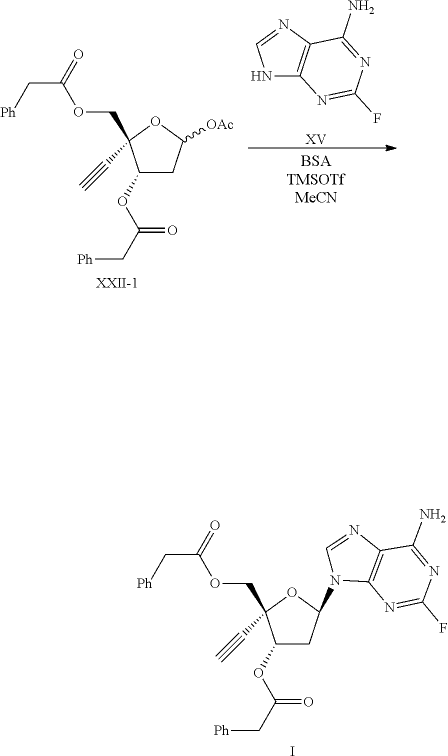

2-Fluoroadenine (XV, 1.1 equiv.), acetonitrile (10.0 volumes) and bis(trimethylsilyl)acetamide (BSA, 3.0 equiv.) were charged to a reactor. The reaction mixture was heated and maintained at about 72° C. for at least 1 hour. The reaction mixture was then cooled to about 20° C. and trimethylsilyl trifluoromethanesulfonate (TMSOTf, 1.1 equiv.) was charged. The reaction mixture was heated to about 72° C., and the solution of crude XXII-1 (1.0 equiv., scaling factor) in acetonitrile was added slowly to the reaction mixture maintaining the reactor contents above 70° C., over about 30 minutes. Additional BSA (1.0 equiv.) was charged, and the reaction mixture was concentrated under vacuum to about 3 volumes and diluted with acetone (6 volumes) and then with water (7 volumes). Organic phase was separated and concentrated under vacuum to an oil. The crude product (a mixture of diasteromers with other byproducts) was subjected to column chromatographic purification to obtain I as a solid.

¹H NMR (400 MHz, Chloroform-d) δ 7.75 (d, J=1.4 Hz, 1H), 7.41-7.19 (m, 11H), 6.45 (s, 1H), 6.34 (t, J=6.7 Hz, 1H), 5.54 (dd, J=6.8, 4.6 Hz, 1H), 4.50 (d, J=12.0 Hz, 1H), 4.41 (d, J=12.0 Hz, 1H), 3.72 (s, 2H), 3.68 (s, 2H), 2.67-2.53 (m, 2H), 2.44 (d, J=0.8 Hz, 1H).

All references, including publications, patents, and patent documents were incorporated by reference herein, as though individually incorporated by reference. The present disclosure provides reference to various embodiments and techniques. However, it should be understood that many variations and modifications may be made while remaining within the spirit and scope of the present disclosure.

1. A process for preparing a compound of formula I: 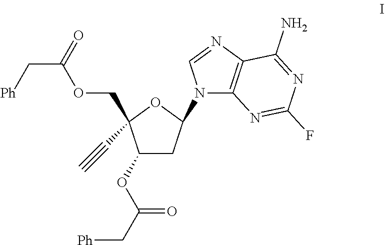 [SMILES: C#C[C@]1(COC(=O)Cc2ccccc2)O[C@@H](n2cnc3c(N)nc(F)nc32)C[C@@H]1OC(=O)Cc1ccccc1]
or a co-crystal, solvate, salt, or combination thereof;
comprising reacting a compound of formula XVII: 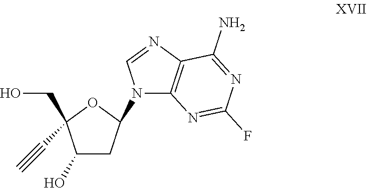 [SMILES: C#C[C@]1(CO)O[C@@H](n2cnc3c(N)nc(F)nc32)C[C@@H]1O]
or a co-crystal, solvate, salt, or combination thereof, with a compound of formula XVIII: 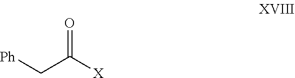
wherein X is selected from halo, —OCOCH₂Ph, and OH, and
a base,
to provide the compound of formula I or a co-crystal, solvate, salt, or combination thereof.

2. The process of claim 1, wherein X is halo.

3. The process of claim 1, wherein X is Cl.

4. The process of claim 1, wherein X is OH, and the reacting is performed in the presence of an activating reagent.

5.-6. (canceled)

7. The process of claim 1, wherein the base comprises an aromatic amine, tertiary alkyl amine, carbonate, or combination thereof.

8. The process of claim 1, wherein the base comprises an aromatic amine.

9.-13. (canceled)

14. The process of claim 1, further comprising preparing the compound of formula XVII or a co-crystal, solvate, salt, or combination thereof by
a process comprising:
reacting a compound of formula XVI-A: 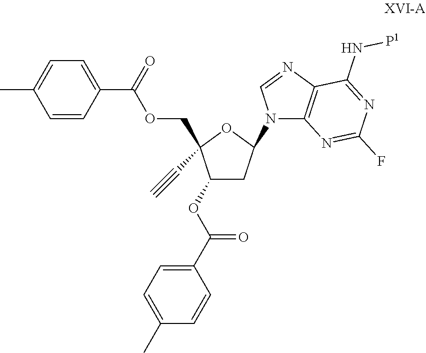
or a co-crystal, solvate, salt, or combination thereof,
wherein P¹ is H or (R⁴)₃Si, wherein each R⁴ is independently C₁₋₆ alkyl, with
a de-acylation reagent, and
a second base,
to provide the compound of formula XVII or a co-crystal, solvate, salt, or combination thereof.

15.-22. (canceled)

23. The process of claim 14, further comprising preparing the compound of formula XVI-A or a co-crystal, solvate, salt, or combination thereof by a process comprising:
reacting a compound of formula XIV: 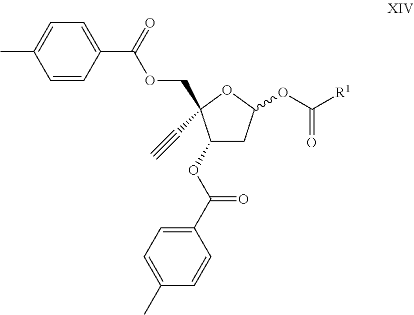
or a co-crystal, solvate, salt, or combination thereof,
wherein R¹ is selected from C₁₋₄ alkyl, C₁₋₄ haloalkyl, C₁₋₄ alkoxy, benzyloxy, or C₆₋₁₀ aryl optionally substituted with 1 to 5 substituents independently selected from C₁₋₄ alkyl, C₁₋₄ haloalkyl, halo, C₁₋₄ alkoxy, or benzyloxy,
with a compound of formula XV: 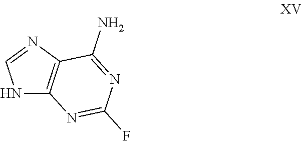 [SMILES: Nc1nc(F)nc2[nH]cnc12]
or a co-crystal, solvate, salt, or combination thereof,
a silylating reagent, and
a Lewis acid,
to provide the compound of formula XVI-A or a co-crystal, solvate, salt, or combination thereof.

24.-30. (canceled)

31. The process of claim 23, further comprising preparing the compound of formula XIV or a co-crystal, solvate, salt, or combination thereof by a process comprising:
reacting a compound of formula XII: 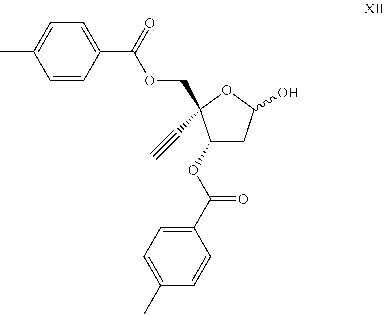 [SMILES: C#C[C@]1(COC(=O)c2ccc(C)cc2)OC(O)C[C@@H]1OC(=O)c1ccc(C)cc1]
or a co-crystal, solvate, salt, or combination thereof, with
a compound selected from the group consisting of a compound of formula XIII-A: 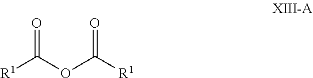
and a compound of formula XIII-B: 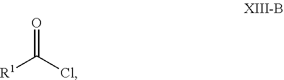
a third base, and
a second catalyst,
to provide the compound of formula XIV or a co-crystal, solvate, salt, or combination thereof.

32.-42. (canceled)

43. The process of claim 31, further comprising preparing the compound of formula XII or a co-crystal, solvate, salt, or combination thereof by a process comprising:
reducing a compound of formula XI: 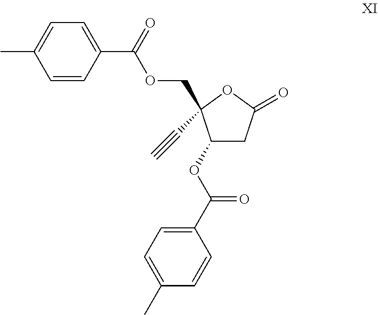 [SMILES: C#C[C@]1(COC(=O)c2ccc(C)cc2)OC(=O)C[C@@H]1OC(=O)c1ccc(C)cc1]
or a co-crystal, solvate, salt, or combination thereof, with
a reducing agent,
to provide the compound of formula XII or a co-crystal, solvate, salt, or combination thereof.

44. (canceled)

45. The process of claim 43, wherein the reducing agent is diisobutylaluminum hydride.

46.-47. (canceled)

48. The process of claim 43, further comprising preparing the compound of formula XI or a co-crystal, solvate, salt, or combination thereof by a process comprising:
esterifying a compound of formula X: 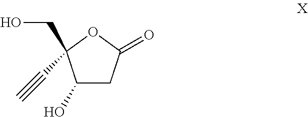
or a co-crystal, solvate, salt, or combination thereof, with
a fourth base,
a third catalyst, and
an acylating reagent,
to provide the compound of formula XI or a co-crystal, solvate, salt, or combination thereof.

49.-61. (canceled)

62. A process for preparing a compound of formula I: 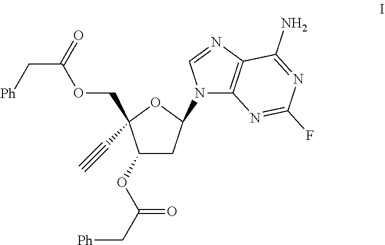 [SMILES: C#C[C@]1(COC(=O)Cc2ccccc2)O[C@@H](n2cnc3c(N)nc(F)nc32)C[C@@H]1OC(=O)Cc1ccccc1]
or a co-crystal, solvate, salt, or combination thereof;
comprising reacting a compound of formula XXII: 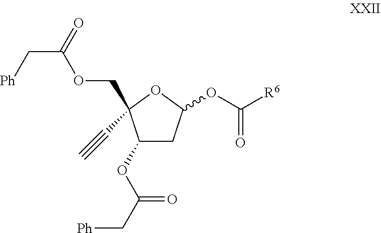
or a co-crystal, solvate, salt, or combination thereof,
wherein R⁶ is selected from C₁₋₄ alkyl, C₁₋₄ haloalkyl, C₁₋₄ alkoxy, benzyloxy, or C₆₋₁₀ aryl optionally substituted with 1 to 5 substituents independently selected from C₁₋₄ alkyl, C₁₋₄ haloalkyl, halo, C₁₋₄ alkoxy, or benzyloxy,
with a compound of formula XV: 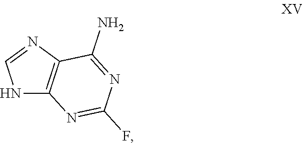 [SMILES: Nc1nc(F)nc2[nH]cnc12]
a silylating reagent, and
a Lewis acid,
to provide the compound of formula I or a co-crystal, solvate, salt, or combination thereof.

63.-96. (canceled)

97. The process of claim 48, further comprising preparing the compound of formula X or a co-crystal, solvate, salt, or combination thereof by a process comprising:
reacting a compound of formula IX: 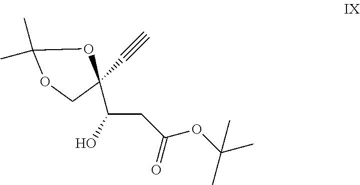 [SMILES: C#C[C@]1([C@@H](O)CC(=O)OC(C)(C)C)COC(C)(C)O1]
or a co-crystal, solvate, salt, or combination thereof, with
an acid,
to provide the compound of formula X or a co-crystal, solvate, salt, or combination thereof.

98.-101. (canceled)

102. The process of claim 97, further comprising preparing the compound of formula IX or a co-crystal, solvate, salt, or combination thereof by a process comprising:
desilylating a compound of formula VIII-A: 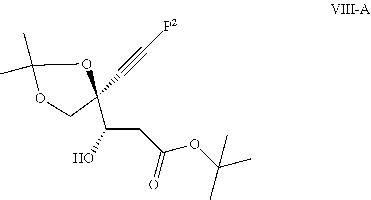
wherein P² is (R⁵)₃Si, wherein each R⁵ is independently C₁₋₆ alkyl;
or a co-crystal, solvate, salt, or combination thereof, with
a fluoride source,
to provide the compound of formula IX or a co-crystal, solvate, salt, or combination thereof.

103.-107. (canceled)

108. The process of claim 102, further comprising preparing the compound of formula VIII-A or a co-crystal, solvate, salt, or combination thereof by a process comprising:
reacting a compound of formula VII-A: 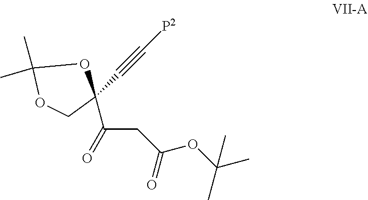
or a co-crystal, solvate, salt, or combination thereof, with
a hydrogen source, and
a fourth catalyst,
to provide the compound of formula VIII-A or a co-crystal, solvate, salt, or combination thereof.

109.-116. (canceled)

117. The process of claim 108, further comprising preparing the compound of formula VII-A or a co-crystal, solvate, salt, or combination thereof by a process comprising:
reacting a compound of formula VI-A: 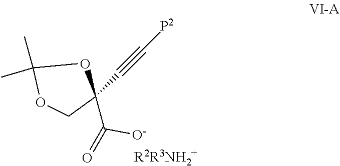
or a co-crystal, solvate, salt, or combination thereof,
wherein R² and R³ are independently selected from C₃-C₇ cycloalkyl, C₁₋₆ alkyl, and benzyl, with
a second acid,
a second activating reagent,
a malonic acid monoester, and
a fifth base,
to provide the compound of formula VII-A or a co-crystal, solvate, salt, or combination thereof.

118.-127. (canceled)

128. The process of claim 117, further comprising preparing the compound of formula VI-A or a co-crystal, solvate, salt, or combination thereof by a process comprising:
oxidizing a compound of formula V-A: 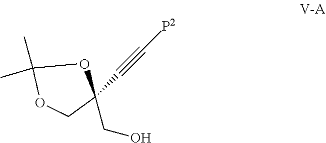
or a co-crystal, solvate, salt, or combination thereof, with
an oxidation reagent,
a fifth catalyst,
a sixth base, and
a salt forming reagent,
to provide the compound of formula VI-A or a co-crystal, solvate, salt, or combination thereof.

129.-138. (canceled)

139. The process of claim 128, further comprising preparing the compound of formula V-A or a co-crystal, solvate, salt, or combination thereof by a process comprising:
reacting a compound of formula IV-A: 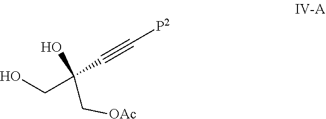
or a co-crystal, solvate, salt, or combination thereof, with
an acid catalyst,
a 1,2-diol protecting reagent, and
a seventh base,
to provide the compound of formula V-A or a co-crystal, solvate, salt, or combination thereof.

140.-147. (canceled)

148. The process of claim 139, further comprising preparing the compound of formula IV-A or a co-crystal, solvate, salt, or combination thereof by a process comprising:
reacting a compound of formula III-A: 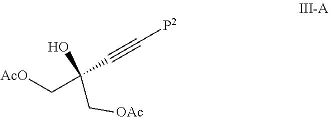
or a co-crystal, solvate, salt, or combination thereof, with
an eighth base, and
an enzyme,
to provide the compound of formula IV-A or a co-crystal, solvate, salt, or combination thereof.

149.-155. (canceled)

156. The process of claim 148, further comprising preparing the compound of formula III-A or a co-crystal, solvate, salt, or combination thereof by a process comprising:
reacting a compound of formula II: 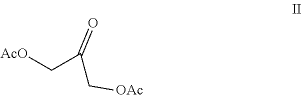 [SMILES: CC(=O)OCC(=O)COC(C)=O]
or a co-crystal, solvate, salt, or combination thereof, with
a compound of formula P²-acetylene, and
a ninth base,
to provide the compound of formula III-A or a co-crystal, solvate, salt, or combination thereof.

157.-161. (canceled)

162. A process for preparing a compound of formula I: 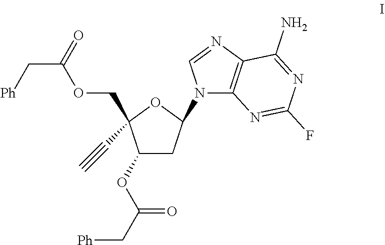 [SMILES: C#C[C@]1(COC(=O)Cc2ccccc2)O[C@@H](n2cnc3c(N)nc(F)nc32)C[C@@H]1OC(=O)Cc1ccccc1]
or a co-crystal, solvate, salt, or combination thereof, comprising:
(a) esterifying a compound of formula X: 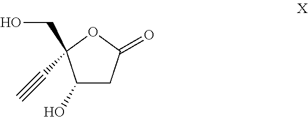
or a co-crystal, solvate, salt, or combination thereof, with
a fourth base,
a third catalyst, and
an acylating reagent,
to provide a compound of formula XI: 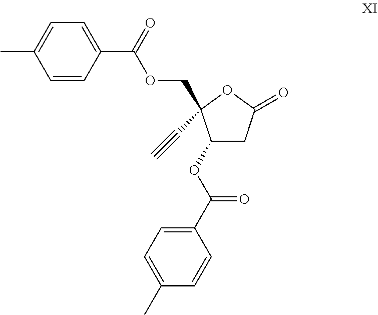 [SMILES: C#C[C@]1(COC(=O)c2ccc(C)cc2)OC(=O)C[C@@H]1OC(=O)c1ccc(C)cc1]
or a co-crystal, solvate, salt, or combination thereof.
(b) reducing the compound of formula XI or a co-crystal, solvate, salt, or combination thereof, with
a reducing agent,
to provide a compound of formula XII: 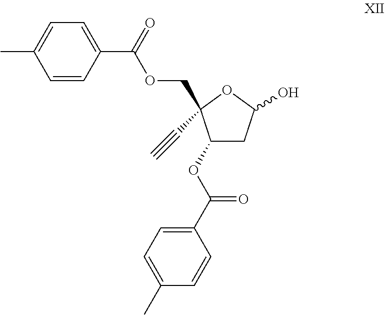 [SMILES: C#C[C@]1(COC(=O)c2ccc(C)cc2)OC(O)C[C@@H]1OC(=O)c1ccc(C)cc1]
or a co-crystal, solvate, salt, or combination thereof;
(c) reacting the compound of formula XII or a co-crystal, solvate, salt, or combination thereof, with
a compound selected from the group consisting of a compound of formula XIII-A: 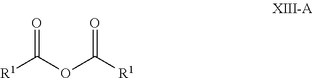
and a compound of formula XIII-B: 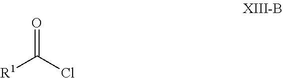
wherein R¹ is selected from C₁₋₄ alkyl, C₁₋₄ haloalkyl, C₁₋₄ alkoxy, benzyloxy, or C₆₋₁₀ aryl optionally substituted with 1 to 5 substituents independently selected from C₁₋₄ alkyl, C₁₋₄ haloalkyl, halo, C₁₋₄ alkoxy, or benzyloxy,
a third base, and
a second catalyst,
to provide a compound of formula XIV: 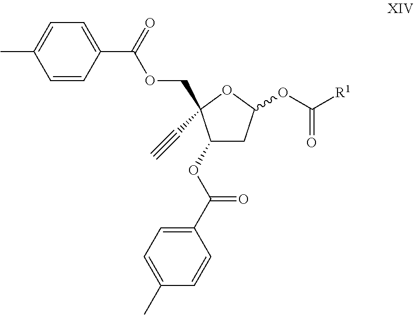
or a co-crystal, solvate, salt, or combination thereof;
(d) reacting the compound of formula XIV or a co-crystal, solvate, salt, or combination thereof, with a compound of formula XV: 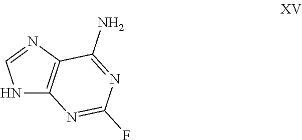 [SMILES: Nc1nc(F)nc2[nH]cnc12]
or a co-crystal, solvate, salt, or combination thereof,
a silylating reagent, and
a Lewis acid,
to provide a compound of formula XVI-A: 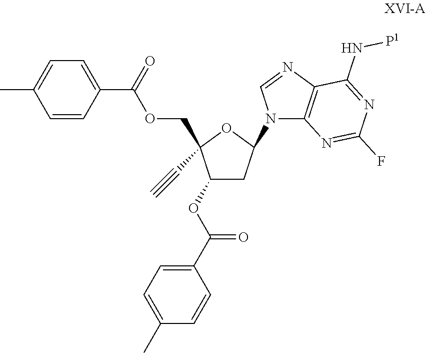
or a co-crystal, solvate, salt, or combination thereof,
wherein P¹ is H or (R⁴)₃Si, wherein each R⁴ is independently C₁₋₆ alkyl;
(e) reacting the compound of formula XVI or a co-crystal, solvate, salt, or combination thereof, with:
a de-acylation reagent, and
a second base,
to provide a compound of formula XVII: 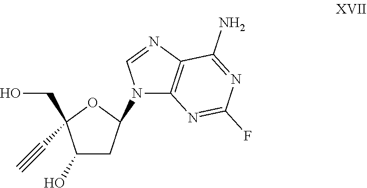 [SMILES: C#C[C@]1(CO)O[C@@H](n2cnc3c(N)nc(F)nc32)C[C@@H]1O]
or a co-crystal, solvate, salt, or combination thereof; and
(f) reacting the compound of formula XVII or a co-crystal, solvate, salt, or combination thereof, with
a compound of formula XVIII: 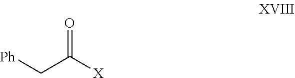
wherein X is selected from halo, anhydride, and OH, and
a base,
to provide the compound of formula I or a co-crystal, solvate, salt, or combination thereof.

163. (canceled)

164. The process of claim 162, wherein the compound of formula X or a co-crystal, solvate, salt, or combination thereof is prepared by a process comprising:
(a) reacting a compound of formula II: 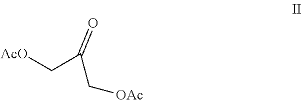 [SMILES: CC(=O)OCC(=O)COC(C)=O]
or a co-crystal, solvate, salt, or combination thereof, with
a compound of formula P²-acetylene, wherein P² is (R⁴)₃Si, wherein each R⁴ is independently C₁₋₆ alkyl, and
a ninth base,
to provide a compound of formula III-A: 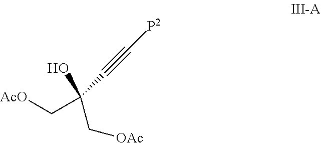
or a co-crystal, solvate, salt, or combination thereof;
(b) reacting the compound of formula III-A or a co-crystal, solvate, salt, or combination thereof, with
an eighth base, and
an enzyme,
to provide a compound of formula IV-A: 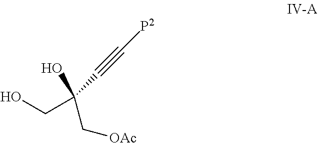
or a co-crystal, solvate, salt, or combination thereof;
(c) reacting the compound of formula IV-A or a co-crystal, solvate, salt, or combination thereof, with
an acid catalyst,
a 1,2-diol protecting reagent, and
a seventh base,
to provide a compound of formula V-A: 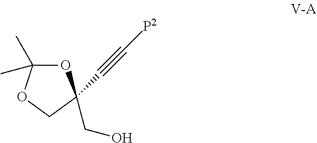
or a co-crystal, solvate, salt, or combination thereof;
(d) oxidizing the compound of formula V-A or a co-crystal, solvate, salt, or combination thereof, with
an oxidation reagent,
a fifth catalyst,
a sixth base, and
a salt forming reagent,
to provide a compound of formula VI-A: 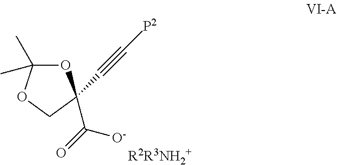
or a co-crystal, solvate, salt, or combination thereof,
wherein R² and R³ are independently selected from C₃-C₇ cycloalkyl, C₁₋₆ alkyl, and benzyl;
(e) reacting the compound of formula VI-A or a co-crystal, solvate, salt, or combination thereof, with
a second acid,
a second activating reagent,
a malonic acid monoester, and
a fifth base,
to provide a compound of formula VII-A: 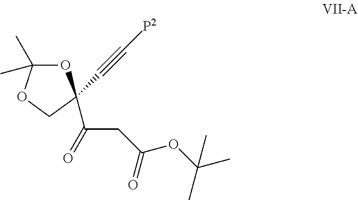
or a co-crystal, solvate, salt, or combination thereof;
(f) reacting the compound of formula VII-A or a co-crystal, solvate, salt, or combination thereof, with
a hydrogen source, and
a fourth catalyst,
to provide a compound of formula VIII-A: 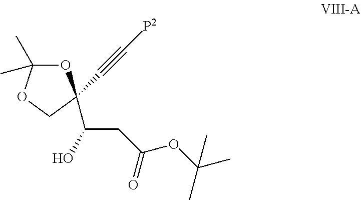
or a co-crystal, solvate, salt, or combination thereof;
(g) desilylating the compound of formula VIII-A or a co-crystal, solvate, salt, or combination thereof, with
a fluoride source,
to provide a compound of formula IX: 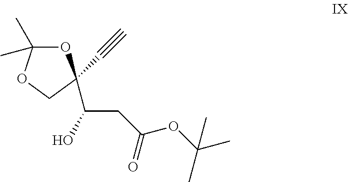 [SMILES: C#C[C@]1([C@@H](O)CC(=O)OC(C)(C)C)COC(C)(C)O1]
or a co-crystal, solvate, salt, or combination thereof; and
(h) reacting the compound of formula IX or a co-crystal, solvate, salt, or combination thereof, with
an acid,
to provide the compound of formula X or a co-crystal, solvate, salt, or combination thereof.

165. A compound selected from the group consisting of a compound of formula XIX: 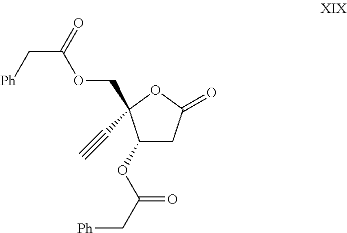 [SMILES: C#C[C@]1(COC(=O)Cc2ccccc2)OC(=O)C[C@@H]1OC(=O)Cc1ccccc1]
or a co-crystal, solvate, salt, or combination thereof; and
a compound of formula XX: 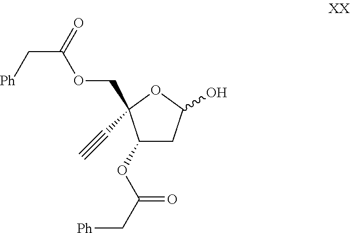 [SMILES: C#C[C@]1(COC(=O)Cc2ccccc2)OC(O)C[C@@H]1OC(=O)Cc1ccccc1]
or a co-crystal, solvate, salt, or combination thereof.

166. (canceled)

167. A compound of formula XXII: 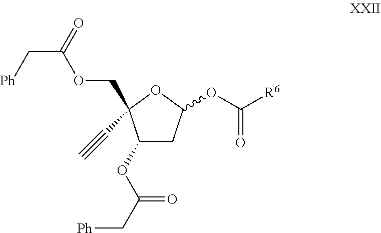
or a co-crystal, solvate, salt, or combination thereof,
wherein R⁶ is selected from C₁₋₄ alkyl, C₁₋₄ haloalkyl, C₁₋₄ alkoxy, benzyloxy, or C₆₋₁₀ aryl optionally substituted with 1 to 5 substituents independently selected from C₁₋₄ alkyl, C₁₋₄ haloalkyl, halo, C₁₋₄ alkoxy, or benzyloxy.

168. (canceled)
